(19) United States
(12) Patent Application Publication
(10) Pub. No.: US 2025/0302865 A1
(43) Pub. Date: Oct. 2, 2025

(54) LECTIN-BINDING CARBOHYDRATES FOR TREATING VIRAL INFECTIONS

(71) Applicant: Pharmalectin, Inc., Needham, MA (US)

(72) Inventor: David PLATT, Newton, MA (US)

(21) Appl. No.: 18/847,466

(22) PCT Filed: Mar. 16, 2023
(86) PCT No.: PCT/US2023/064511

§ 371 Date: Sep. 16, 2024

Related U.S. Application Data
(60) Provisional application No. 63/320,544, filed on Mar. 16, 2022.

(51) Int. Cl. A61K 31/732; A61P 31/14

(52) U.S. Cl. CPC A61K 31/732; A61P 31/14

(57) ABSTRACT

The invention provides a method for treating a viral infection in a subject in need thereof by administering an effective amount of lectin-binding carbohydrates to the subject.

### BACKGROUND OF THE INVENTION

Infections caused by viruses are a burden on global public health. For example, the ongoing SARS-CoV-2 pandemic has caused more than 5,500,000 deaths, and HIV/AIDS causes approximately one million deaths per year. Additional treatments for viral infections are needed.

### SUMMARY OF THE INVENTION

The present invention provides a method of treating a viral infection in a subject in need thereof, by administering to the subject an effective amount of lectin-binding carbohydrates.

In some embodiments of the method of the invention, the lectin-binding carbohydrates are pectin polysaccharides. In some embodiments of the method of the invention, the lectin-binding carbohydrates are fruit pectin polysaccharides.

In some embodiments of the method of the invention, the lectin-binding carbohydrates are galactomannans. In some embodiments, the lectin-binding carbohydrates are one of or a mixture of any combination of fenugreek galactomannans, guar galactomannans, tara galactomannans, locust bean gum galactomannans, and cassia gum galactomannans. In some embodiments, the lectin-binding carbohydrates are a mixture of fenugreek galactomannans and guar galactomannans.

In some embodiments of the method of the invention, the lectin-binding carbohydrates are polysaccharides that include N-acetylglucosamine and mannose. In some embodiments, the lectin-binding carbohydrates further include galactose and/or N-acetylneuraminic acid.

In some embodiments of the invention, the lectin-binding carbohydrates are glycosaminoglycans. In some embodiments, the lectin-binding carbohydrates are selected from the group consisting of heparin, heparin sulfate, chondroitin sulfate, dermatan sulfate, keratan sulfate, and hyaluronan.

In some embodiments of the method of the invention, the lectin-binding carbohydrates are glycolipids. In some embodiments, the lectin-binding carbohydrates are glycosphingolipids. In some embodiments, the lectin-binding carbohydrates are selected from the group consisting of cererosides, gangliosides, and globosides.

In some embodiments of the method of the invention, the lectin-binding carbohydrates are polylactosamines.

In some embodiments of the method of the invention, the lectin-binding carbohydrates include sialic acid.

In some embodiments of the method of the invention, the viral infection is caused by a retrovirus. In some embodiments, the viral infection is caused by human immunodeficiency virus, human T-lymphotropic virus type 1, or human T-lymphotropic virus type 2.

In some embodiments of the method of the invention, the viral infection is a human norovirus infection.

In some embodiments of the method of the invention, the viral infection is caused by a human herpesvirus. In some embodiments, the viral infection is caused by herpes simplex virus 1, herpes simplex virus 2, varicella zoster virus, human cytomegalovirus, Epstein-Barr virus, roseolovirus, pseudorabies virus, or Kaposi's sarcoma associated herpesvirus.

In some embodiments of the method of the invention, the viral infection is caused by a coronavirus. In some embodiments, the viral infection is caused by SARS-CoV-2, SARS-CoV-1, MERS-CoV, human coronavirus 229E, human coronavirus NL63, human coronavirus OC43, or human coronavirus HKU1. In some embodiments, the viral infection is caused by SARS-CoV-2.

In some embodiments of the method of the invention, the viral infection is caused by an orthomyxovirus. In some embodiments, the viral infection is caused by an alphainfluenzavirus, betainfluenzavirus, deltainfluenzavirus, gammainfluenzavirus, isavirus, thogotovirus, or quaranjavirus.

In some embodiments of the method of the invention, the viral infection is caused by an adenovirus. In some embodiments, the viral infection is caused by human adenovirus A, human adenovirus B, human adenovirus C, human adenovirus D, human adenovirus E, human adenovirus F, or human adenovirus G.

In some embodiments of the method of the invention, the viral infection is caused by a flavivirus. In some embodiments, the viral infection is caused by the West Nile virus, dengue virus, tick-born encephalitis virus, yellow fever virus, Zika virus, hepatitis C virus, Murray Valley encephalitis virus, Tick-borne encephalitis virus, Saint Louis encephalitis virus, or Japanese encephalitis virus.

In some embodiments of the method of the invention, the viral infection is caused a rotavirus. In some embodiments, the viral infection is caused by rotavirus A, rotavirus B, rotavirus C, rotavirus D, rotavirus F, rotavirus G, rotavirus H, rotavirus I, or rotavirus J.

In some embodiments of the method of the invention, the viral infection is caused a mononegavirus. In some embodiments of the method of the invention, the viral infection is caused an orthopneumovirus. In some embodiments, the viral infection is caused by human metapneumovirus, human respiratory syncytial virus A2, or human respiratory syncytial virus B1.

In some embodiments, the viral infection is caused by the Ebola virus, Marburg virus, measles virus, mumps virus, Nipah virus, or rabies virus.

In some embodiments of the method of the invention, the viral infection is a rubella virus infection.

In some embodiments of the method of the invention, the lectin-binding carbohydrates are formulated in a pharmaceutical composition with a pharmaceutically acceptable carrier. In some embodiments, the pharmaceutical composition is a solid oral dosage form. In some embodiments, the solid oral dosage form is a chewable tablet. In some embodiments of the invention, the pharmaceutical composition is for intravenous administration.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the term “approximately” may be understood to permit standard variation as would be understood by those of ordinary skill in the art; and (v) where ranges are provided, endpoints are included.

As used herein, the term “about” represents a value that is in the range of ±10% of the value that follows the term “about.” Reference to “about” a value or parameter herein includes (and describes) variations that are directed to that value or parameter per se. For example, description referring to “about X” includes description of “X”.

As used herein, the term “administration” refers to the administration of a composition (e.g., a compound or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, or vitreal.

An “effective amount” of a compound may vary according to factors such as the disease state, age, sex, and weight of the individual, and the ability of the compound to elicit the desired response. A therapeutically effective amount encompasses an amount in which any toxic or detrimental effects of the compound are outweighed by the therapeutically beneficial effects. An effective amount also encompasses an amount sufficient to confer benefit, e.g., clinical benefit.

As used herein, the term “host cell” refers to a cell that is entered by a virus during a viral infection.

As used herein, the terms “treat,” “treated,” or “treating” mean both therapeutic treatment and prophylactic or preventive measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the subject; or enhancement or improvement of condition, disorder, or disease. Treatment includes eliciting a clinically significant response without excessive levels of side effects. Treatment also includes prolonging survival as compared to expected survival if not receiving treatment.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF THE DRAWINGS

FIG. 1 is a plot showing the reduction of viral load (circles) and viral particles (log 10, squares) in Vero cells pre-treated with Prolectin-M (PL-M).

FIG. 2 is a plot showing the reduction of viral load (circles) and viral particles (log 10, squares) in Vero cells pre-treated with PL-M.

FIG. 3 is a plot showing non-linear regression curves showing the reduction of viral load as a function of PL-M (squares) and Prolectin-I (PL-I) (circles) concentrations using Protocol 1. For PL-M and PL-I, EC₅₀ and EC₉₀ values (which are essentially the same) are 1.53 μg/ml and 9.26 μg/ml, respectively.

FIG. 4 is a plot showing non-linear regression curves showing the reduction of viral presence as a function of PL-M (squares) and PL-I (circles) concentrations using Protocol 2. For PL-M, IC₅₀=6.18 μg/ml, whereas for PL-I IC₅₀=4.25 μg/ml.

FIG. 5 is a bar graph showing percentage of viral copies (black bars) and viral copy reduction (grey bars) using PL-M.

FIG. 6 is a bar graph showing percentage of viral copies (black bars) and viral copy reduction (grey bars) using PL-I. All data represent the mean±SD of triplicate experiments.

FIG. 7 is a plot showing ¹⁵N HSQC expansions that are overlaid for ¹⁵N-labeled Gal-3 (20 μM) in the absence and presence of 1.2 mg/ml PL-M.

FIG. 8 is a chemical shift map (Δδ vs. the amino acid sequence of Gal-3) is shown for the binding of PL-M to Gal-3. Chemical shifts were internally referenced to DSS (4,4-dimethyl-4-silapentane-1-sulfonic acid), and chemical shift differences (Δδ) were calculated as [(Δ¹H)²+(0.25Δ¹⁵N)²]^{1/2}. Solution conditions were 20 mM potassium phosphate, pH 6.9.

FIG. 9 is the crystal structure of the Gal-3 CRD (pdb access code: 1A3K; Seetharaman et al., 1998) shown with the largest Δδ values highlighted in red (>2SD above the Δδ average), orange (between 1SD to 2SD above the Δδ average), yellow (between the average and 1SD above the Δδ average), and aqua (below the Δδ average). For orientation, a molecule of bound lactose is shown in dark blue in stick format.

FIG. 10 is a plot showing the Ao values averaged over all Gal-3 residues plotted vs the concentration of PL-M. Data were exponentially fitted as show by the dashed line.

FIG. 11 is a plot showing the reduction of viral load and viral particles in Vero cells pre-treated with PL-I (Protocol 1). Viral particles were reduced from 10^{6.8} to 10^{5.1}.

FIG. 12 is a plot showing the reduction of viral load and viral particles in Vero cells post-treated with PL-I (Protocol 2). Viral particles were reduced from 10^{6.8} to 10^{5.2}.

FIG. 13 is a plot showing that no cytotoxic effects were observed by PL-M on Vero cells. PL-M increased the percent of viable cells: CC₅₀=>100 μg/ml. All data represent the mean±SD of triplicate experiments).

FIG. 14 is a plot showing that no cytotoxic effects were observed by PL-I on Vero cells. PL-M increased the percent of viable cells: CC₅₀=>100 μg/ml. All data represent the mean±SD of triplicate experiments).

FIG. 15 is a total ion chromatogram showing the results of gas chromatography/mass spectrometry analysis of partially methylated alditol acetate derivatives made from a sample of galactomannans.

FIG. 16 is a ¹H-NMR spectrum of the anomeric region of galactomannans.

FIG. 17 is a full range ¹H-NMR spectrum of galactomannans.

FIG. 18 is a HSQC spectrum of the anomeric region of galactomannans.

FIG. 19 is a HSQC spectrum of the glycosyl ring H/C region of galactomannans.

FIG. 20 is a TOCSY spectrum of galactomannans.

FIG. 21 is a NOESY spectrum of galactomannans.

FIG. 22 shows the structure of galactomannans employed in the present invention.

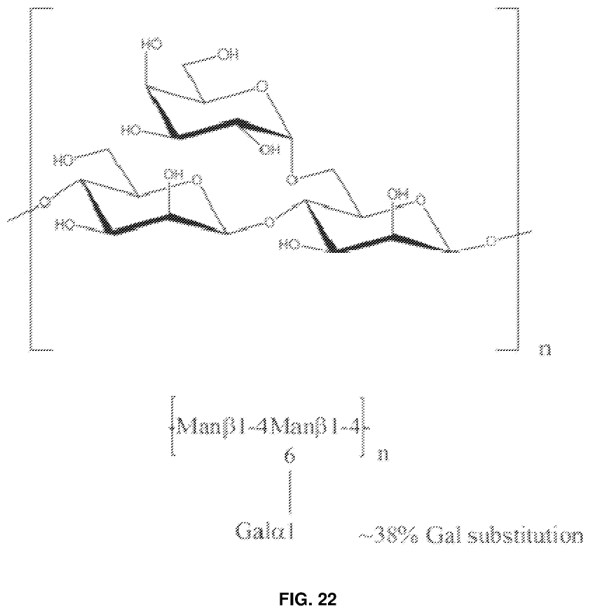

FIG. 23 shows the structure of galactomannans employed in the present invention.

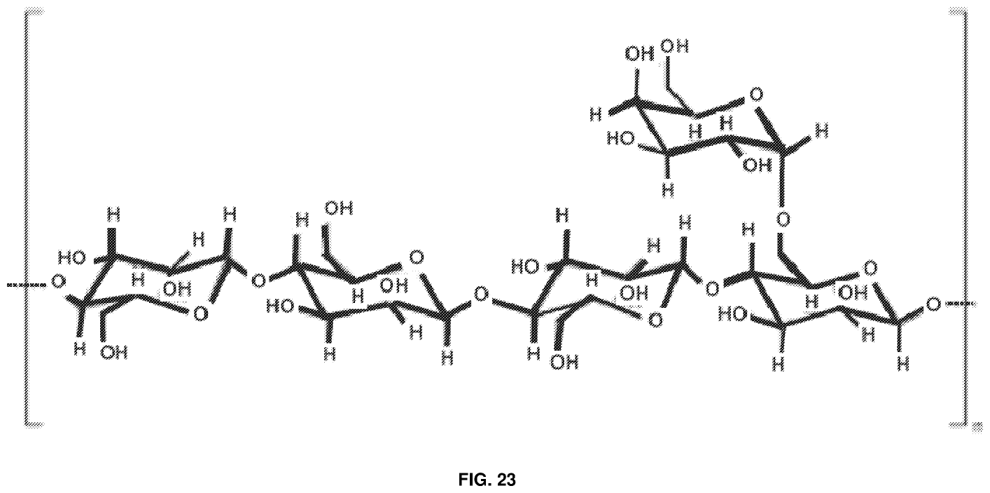

FIG. 24 shows the structure of galactomannans employed in the present invention.

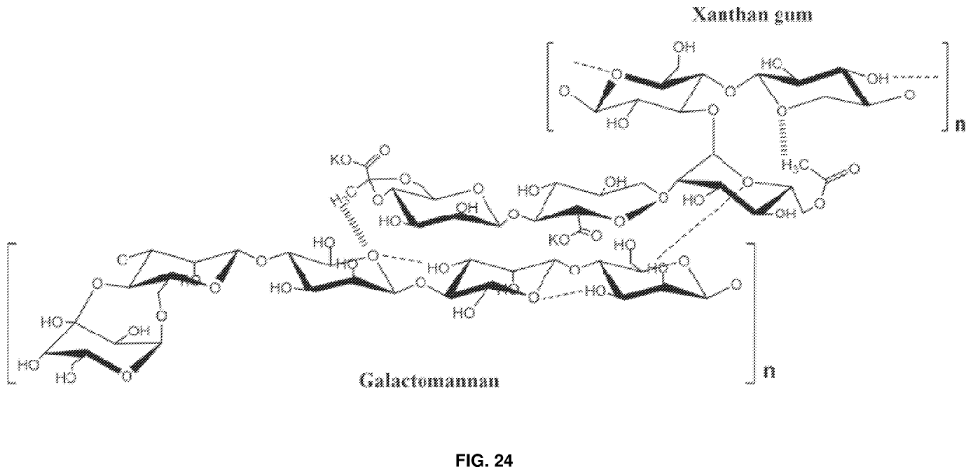

FIG. 25 shows the structure of galactomannans employed in the present invention.

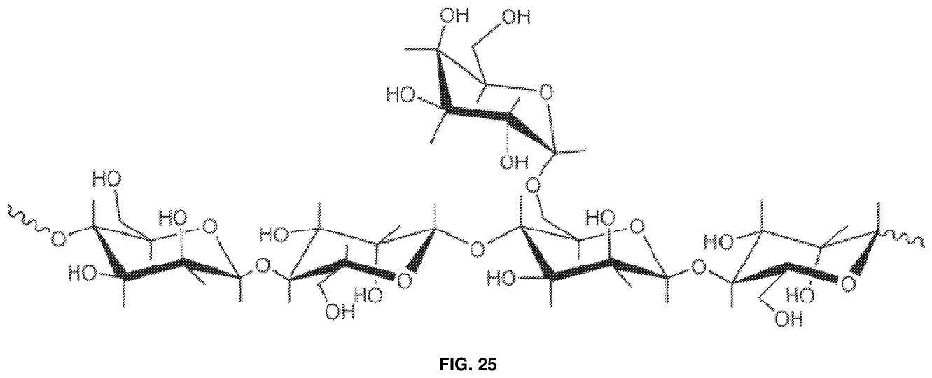

FIG. 26 is a total ion chromatograph showing the results of gas chromatography/mass spectrometry analysis of partially methylated alditol acetate derivatives made from a sample of pectin polysaccharides.

FIG. 27 is a full range ¹H NMR spectrum of pectin polysaccharides.

FIG. 28 is a ¹H NMR spectrum of the carbohydrate region of pectin polysaccharides.

FIG. 29 is a HSQC spectrum of the anomeric region of pectin polysaccharides.

FIG. 30 is a HSQC spectrum of the glycosyl ring-H/C region of pectin polysaccharides.

FIG. 31 is a TOCSY spectrum of pectin polysaccharides.

FIG. 32 shows the structure of pectin polysaccharides employed in the present invention.

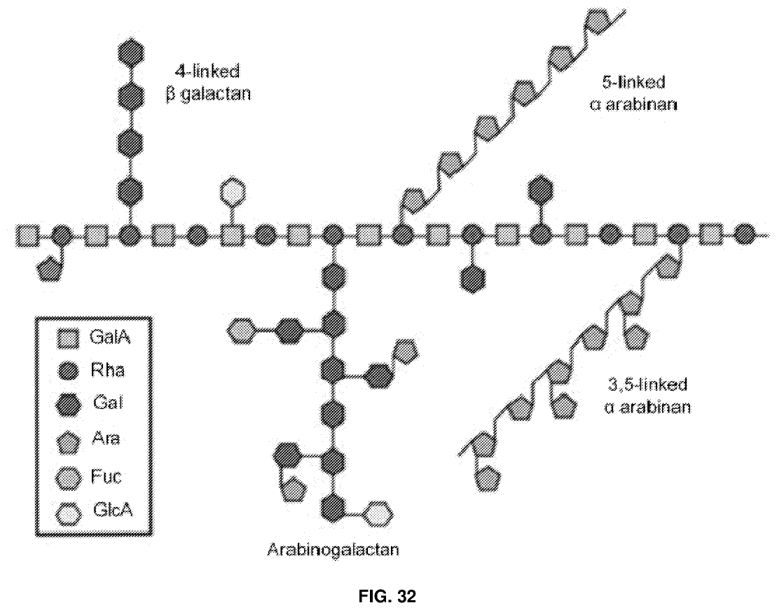

FIG. 33 shows the structure of pectin polysaccharides employed in the present invention.

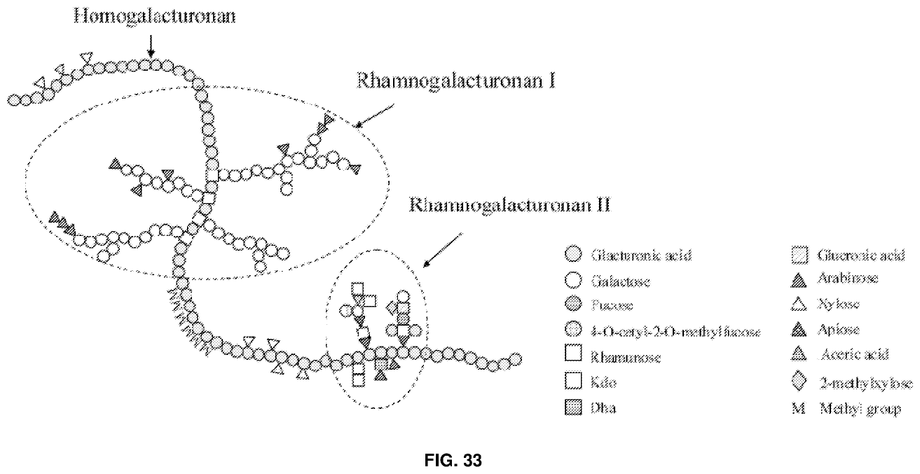

### DETAILED DESCRIPTION

The present inventors have discovered that viruses use carbohydrates that bind to lectins in order to enter cells, and that the administration of exogenous lectin-binding carbohydrates to a subject is deleterious to the ability of the virus to enter host cells of the subject. Therefore, one object of this invention is to provide a method for treating viral infections by administering an effective amount of lectin-binding carbohydrates to a subject in need thereof.

### Lectin-Binding Carbohydrates

All carbohydrates that bind to lectins are embraced by the methods of this invention. Binding between carbohydrates and lectins may be established by any technique known in the art to be useful for the detection of an interaction between a protein and a protein binding partner, including but not limited to nuclear magnetic resonance (NMR) spectroscopy, gel-shift chromatography, cell or protein adhesion assays, fluorescence anisotropy, and isothermal titration calorimetry. Naturally occurring lectin-binding carbohydrates are commonly covalently linked to glycoproteins. This invention contemplates the administration of lectin-binding carbohydrates that are not linked in a glycoprotein and the administration of lectin-binding carbohydrates that are linked in glycoproteins. Illustrative and non-limiting examples of lectin-binding carbohydrates are described below.

### Pectin Polysaccharides

Pectin polysaccharides are complex, heterogeneous, glycans that can be derived from crude biomass and that include terminal arabinofuranosyl residues, terminal arabinopyranosyl residues, 2-linked rhamnopyranosyl residues, terminal galactopyranosyl residues, terminal galactopyranosyl uronic acid residues, 2-linked xylopyranosyl residues, 4-linked xylopyranosyl residues, 2,4-linked rhamnopyranosyl residues, 2,4-linked rhamnopyranosyl residues, 3-linked galactopyranosyl residues, 4-linked galactopyranosyl residues, 4-linked galactopyranosyl uronic acid residues, 4-linked glucopyranosyl residues, 3,4-linked galactopyranosyl uronic acid residues, and/or 3,5-linked galactopyranosyl residues.

Pectin polysaccharides may be obtained via the processing of crude fruit pectins, e.g., apple pectins, e.g., pectins derived from apple pomace, or citrus pectins, e.g., pectins derived from citrus peels, e.g., the peels of oranges, lemons, or limes, or from the processing of soybean pectins, e.g., pectins derived from soybean hulls, or sugar beet pectins, e.g., pectins derived from sugar beets. In some embodiments, Pectin polysaccharides are derived from apple pomace. In some embodiments, the pectin polysaccharides is obtained through chemical, enzymatic, physical treatment, and purification from pectic substance of citrus peels and apple pomace or soybean hull or alternatively processed from sugar beet pectin, e.g., as described in U.S. Pat. No. 10,744,154, which is hereby incorporated by reference. An exemplary pectin polysaccharide is Prolectin-I, as described herein.

Although the composition of pectin may vary among plants, pectin typically has a composition in which D-galacturonic acid is the main monomeric constituent. The D-galacturonic residues of pectin optionally may be substituted with D-xylose or D-apiose to form xylogalacturonan and apiogalacturonan, respectively, branching from a D-galacturonic acid residue. So-called “rhamnogalcturonan pectins” contain a backbone of repeating disaccharides of D-galacturonic acid and L-rhamnose.

In some embodiments, pectin polysaccharides are prepared by modifying naturally occurring polymers to reduce the molecular weight for the desired range, reducing the alkylated group (de-methoxylation or deacetylation). Prior to chemical modification, the natural polysaccharides may have a molecular weight range of between about 40,000-1,000,000 Da with multiple branches of saccharides, for example, branches including 1 to 20 monosaccharides of glucose, arabinose, galactose etc, and these branches may be connected to the backbone via neutral monosaccharides such as rhamnose. These molecules may further include a single or chain of uronic acid saccharide backbone that may be esterified from as little as about 2% to as much as about 30%. The multiple branches themselves may have multiple branches of saccharides, the multiple branches optionally including neutral saccharides and neutral saccharide derivatives creating mainly hydrophobic entities.

In some embodiments, pectin polysaccharides have a weight-average molecular weight of about 40 kDa to about 1 MDa, e.g., 50 kDa to about 500 kDa, about 60 kDa to about 400 kDa, about 70 kDa to about 300 kDa, about 80 kDa to about 200 kDa, about 90 kDa to about 150 kDa, about 100 kDa to about 140 kDa, about 110 kDa to about 130 kDa, or about 120 kDa. In some embodiments, pectin polysaccharides have a weight average molecular weight of about 120 kDa.

In some embodiments, pectin polysaccharides have a heterogeneous structure with five principal components: rhamnose, fucose, arabinose, galactose, and uronate. In some embodiments, pectin polysaccharides are about 1% to about 10% rhamnose by weight, e.g., about 2% to about 8%, about 3% to about 7%, about 4% to about 6%, about 4.3% rhamnose by weight; about 1% to about 10% fucose by weight, e.g., about 2% to about 6%, about 3% to about 5%, about 3.7% fucose by weight; about 10% to about 30% arabinose by weight, e.g., about 12% to about 28%, about 14% to about 26%, about 16% to about 24%, about 18% to about 22%, about 19% arabinose by weight; about 30% to about 50% galactose by weight, e.g., about 32% to about 46%, about 34% to about 42%, about 36% to about 48%, about 37% galactose by weight; and about 25% to about 45% uronate by weight, e.g., about 27% to about 43%, about 29% to about 41%, about 31% to about 39%, about 33% to about 37%, about 36% uronate by weight. In some embodiments, the backbone of pectin polysaccharides is mainly composed of α-(1,2)-L-rhamnosyl-α-(1,4)-D-galacturonosyl sections.

In some embodiments, pectin polysaccharides are a branched heteropolymer of alternating alpha-1,2-linked rhamnose and alpha-1,4-linked galacturonic acid residues that carries neutral side-chains of predominantly 1,4-beta-D-galactose and/or 1,5-alpha-L-arabinose residues attached to the rhamnose residues of the RGI backbone. RGI side-chains may be decorated with arabinosyl residues (arabinogalactan I) or other sugars, including fucose, xylose, and mannose.

### Galactomannans

Galactomannans are polysaccharides derived from plant biomass containing mannose or galactose moieties, or both groups, as the main structural components. The galactomannans described herein are a mixture of complex carbohydrates and include (1-6)-alpha-D-mannopyranosyl, 4-linked mannopyranosyl residues, 6-linked mannopyranosyl residues, 4-linked galactopyranosyl residues, 6-linked galactopyranosyl residues, 4-linked glucopyranosyl residues, 6-linked glucopyranosyl residues, 4,6-linked mannopyranosyl residues, 4,6-linked glucopyranosyl residues, terminal mannopyranosyl residues, terminal glucopyranosyl residues, and/or terminal galactopyranosyl residues. In some embodiments, the galactomannans described herein include linear chains of (1-4)-beta-D-mannopyranosyl units with alpha-D-galactopyranosyl units attached by 1-6 linkages. The carbohydrates may be in the range of 500-1000 D, 10 kD to 50 kD (e.g., 20 kD-40 kD), and/or 50-500 kD. In preferred embodiments, the galactomannans are water soluble.

Exemplary sources of galactomannans are one or more of Trigonella foenum-graecum, Cyamopsis tetragonoloba, Acacia senegal, Acacia seyal, Ceratonia siliqua, Cassia fistula, Senna obtusifolia, Senna tora, and Caesalpinia spinosa. In some embodiments, the galactomannans are one or more of fenugreek (e.g., from Trigonella foenum-graecum) galactomannans; guar (e.g., from Cyamopsis tetragonoloba) galactomannans; tara (e.g., from Caesalpinia spinosa or Tara spinosa) galactomannans; locust bean gum (e.g., from Ceratonia siliqua) galactomannans; and cassia gum (e.g., from Senna obtusifolia or Senna tora) galactomannans. In some embodiments, the galactomannans include gum acacia (e.g., from Acacia senegal or Acacia seyal).

In some embodiments, the galactomannans are a mixture of any combination of fenugreek galactomannans, guar galactomannans, tara galactomannans, locust bean gum galactomannans, and cassia gum galactomannans. An exemplary galactomannan is Prolectin-M, as described herein. An exemplary composition of Prolectin-M is described is described below:

### Chemical Identification

Substance Name: Guar Gum
Chemical Classification category: Carbohydrates and derivatives
Catalog Name: Chewable tablets composed mainly of GRAS grade simple and complex carbohydrates.

### Composition:

Main Ingredient: Mannans
Synonyms: Soluble mannans, polysaccharides purified from seeds of the plants Trigonella foenum-graecum, Cyamopsis tetragonoloba, Acacia senegal, Acacia seyal and/or Ceratonia siliqua.
CAS #: 9000-30-0
EC #: 232-536-8
Other Ingredients: Food grade sorbitol, erythritol, malic acid, natural flavor and colors.

### Manufacturing/Use Information

Both finished products contained 3 g of mannans in a single dose (two chewable tablets or one succulent).

In some embodiments, the galactomannans are chemically modified. For example, hydroxyethyl, hydroxypropyl and carboxymethylhydroxypropyl substitutions may be made to the galactomannans of the present invention. Non-ionic modifications to the galactomannans, such as those containing alkoxy and alkyl (C₁-C₆) groups, may be made to the galactomannans of the present invention. Anionic substitution may also be made to the galactomannans of the present invention.

In some embodiments, the galactomannans include at least one polysaccharide of high molecular weight and at least one polysaccharide of low molecular weight. In some embodiments, galactomannans include at least one polysaccharide of high molecular weight, at least one polysaccharide of low molecular weight, and at least one oligosaccharide, monosaccharide, and/or sugar alcohol.

In some embodiments, the polysaccharide of low molecular weight has a molecular weight of about 5-50 kDa, e.g., about 10-40 kDa, about 15-35 kDa, or about 20-30 kDa. In some embodiments, the polysaccharide of high molecular weight has a molecular weight of about 20-300 kDa, e.g., about 25-200 kDa, about 35-150 kDa, or about 50-100 kDa.

The one or more oligosaccharides, monosaccharides, and/or sugar alcohols may include, but are not limited to, galacturonic acid, galactose, mannose, mannitol, erythritol, sorbitol, inositol, raffinose (a nonreducing trisaccharide), galactinol (dulcitol), stachyose, verbascose, manninotriose, and higher homologs. In some embodiments, the oligosaccharides, monosaccharides, and/or sugar alcohols have a molecular weight of approximately 500-1,000 Da, e.g., about 600-800 Da, or about 650-700 Da.

In some embodiments, the galactomannans include about 1 part of the at least one polysaccharide of high molecular weight, about 2 parts of the at least one purified mannan polysaccharide of low molecular weight, and about 1 part of oligosaccharides, monosaccharides, and/or sugar alcohol.

In some embodiments of the present invention, the galactomannans may vary in the composition of its constituent carbohydrates. In some embodiments, the constituent carbohydrates vary in the ratio of galactose to mannose. Specifically, they may include about 95% galactose and about 5% mannose, about 90% galactose and about 10% mannose, about 80% galactose and about 20% mannose, about 70% galactose and about 30% mannose, about 60% galactose and about 40% mannose, about 50% galactose and about 50% mannose, about 40% galactose and about 60% mannose, about 30% galactose and about 70% mannose, about 20% galactose and about 80% mannose, about 10% galactose and about 90% mannose, less than about 5% galactose and greater than about 95% mannose, greater than 95% galactose and less than 5% mannose, greater than 90% galactose and less than 10% mannose, greater than 80% galactose and less than 20% mannose, greater than 70% galactose and less than 30% mannose, greater than 60% galactose and less than 40% mannose, greater than 50% galactose and less than 50% mannose, greater than 40% galactose and less than 60% mannose, greater than 30% galactose and less than 70% mannose, greater than 20% galactose and less than 80% mannose, greater than 10% galactose and less than 90% mannose, greater than 5% galactose and less than 95% mannose, 95%±5% galactose and 5%±0.5% mannose, 90%±9% galactose and 10%±1% mannose, 80%±8% galactose and 20%±2% mannose, 70%±7% galactose and 30%±3% mannose, 60%±6% galactose and 40%±4% mannose, 50%±5% galactose and 50%±5% mannose, 40%±4% galactose and 60%±6% mannose, 30%±3% galactose and 70%±7% mannose, 20%±2% galactose and 80%±8% mannose, 10%±1% galactose and 90%±9% mannose, less than 5%±0.5% galactose and greater than 95%±5% mannose.

Polysaccharides with N-Acetylglucosamine and Mannose

Carbohydrates that are covalently linked to the surface of host cells, e.g., carbohydrates that are covalently linked to glycoproteins on the surface of host cells, are commonly linked to the glycoprotein by a core region that comprises the carbohydrates N-acetylglucosamine and mannose. Additional carbohydrates, including but not limited to galactose, N-acetylneuraminic acid, mannose and N-acetylglucosamine, may be bound to the core region.

### Glycolipids

Glycolipids are lipid molecules in which a carbohydrate is covalently linked to the polar head group of an amphipathic lipid. Glycolipids are expressed in the membranes of eukaryotic cells and can facilitate viral entry into host cells by binding to lectins expressed on the surface of viruses. Nonlimiting examples of the carbohydrates that are covalently linked to the polar head group of the lipids are N-acetyl galactosamine, N-acetyl glucosamine, galactose, and glucose. Glycosphingolipids are a group of lipid molecules in which carbohydrates are covalently linked to polar head group of a sphingolipid, e.g., a ceramide.

### Polylactosamines

Polylactosamines are multimeric carbohydrates, e.g., oligomeric carbohydrates and polymeric carbohydrates, that comprise multiple N-acetyllactosamine monomers. Polylactosamines are commonly covalently linked to eukaryotic glycoproteins and are bound by lectins.

Carbohydrates that Comprise Sialic Acids

Sialic acids are a class of alpha-keto acid carbohydrates that are widely expressed on the surface of eukaryotic cells. Exemplary and nonlimiting examples of sialic acids include neuraminic acid and 2-keto-3-deoxynonic acid. Sialic acid multimers and monomers are expressed on the surface of host cells and bind to viral lectins.

### Glycosaminoglycans

Glycosaminoglycans are linear polysaccharide chains that contain repeated disaccharide subunits of (1) carbohydrate that comprises a carboxylic acid group, e.g., a uronic acid and (2) a carbohydrate that comprises an animo group, e.g., an amino carbohydrate. Glycosaminoglycans are commonly substituted with sulfate groups and, in biology, may be covalently linked to glycoproteins, e.g., on the surface of a cell, e.g., a host cell. Glycosaminoglycan identity is determined by the identity of the carbohydrates that make up the repeating disaccharide unit. For example, hyaluronan contains repeating units of glucuronic acid and N-acetylglucosamine; heparin contains repeating units of iduronic acid and N-acetylglucosamine, heparin sulfate contains repeating units of sulfated iduronic acid and sulfated N-acetylglucosamine, chondroitin sulfate contains repeating units of sulfated glucuronic acid and sulfated N-acetylglucosamine, dermatan sulphate contains repeating units of sulfated iduronic acid and sulfated N-acetylglucosamine, and keratan sulfate contains repeating units of galactose and N-acetylglucosamine. This invention contemplates the use of all glycosaminoglycans.

### Glycoproteins

Glycoproteins are proteins in which a plurality of the side chains of the proteins' constituent amino acids are covalently linked to a carbohydrate (e.g., a monomeric carbohydrate, an oligomeric carbohydrate, a polymeric carbohydrate). The carbohydrates that are covalently linked to the amino acid side chains are referred to as glycans. In some embodiments, the glycans are linked to an oxygen atom in the side chain of a serine or threonine amino acid (i.e., the carbohydrates are O-linked glycans). In some embodiments, the glycans are linked to a nitrogen atom in the side chain of an asparagine or lysine amino acid (i.e., the glycans are N-linked glycans). Glycoproteins may be expressed on the surface of host cells or viruses. Glycoproteins that are expressed on the surface of host cells may bind to lectins expressed on the surface of viruses, and glycoproteins that are expressed on the surface of viruses may bind to lectins that are expressed on the surface of host cells.

### Lectins

Lectins are proteins that non-covalently bind to carbohydrates, e.g., at a conserved carbohydrate binding site or pocket.

In some embodiments of the method of the present invention, the lectins are expressed by a host cell and embedded in the membrane of the host cell (membrane-associated host lectins). In some embodiments, lectins are expressed by a host cell and transported outside of the host cell (soluble lectins). Lectins facilitate the entry of viruses into host cells by binding to glycoproteins that are expressed by viruses.

In some embodiments of the method of the present invention, the lectins are encoded and expressed by viruses. Virally-expressed lectins assist in the proliferation of a viral infection by binding to carbohydrates that are expressed on the surface of a host cell, e.g., carbohydrates that are bound to a glycoprotein that is expressed on the surface of a host cell. A nonlimiting example of a lectin that is encoded and expressed by a virus is hemagglutinin, which is a lectin expressed by the Influenza A virus.

In some embodiments of the method of the present invention, the lectins are galectins. Galectins are lectins that that contain a conserved carbohydrate-binding domain and bind to β-galactoside carbohydrates such as N-acetyllactosamine or multimers thereof (i.e., polylactosamines). Carbohydrates that expressed on the surface of viruses, e.g., carbohydrates that are bound to viral glycoproteins, may bind to galectins. Galectins 1, 2, 3, 4, 7, 7B, 9, 9B, 9C, 10, 12, 13, 14, and 16 have been identified in humans.

### Viruses

Methods of the invention may be used to treat a variety of viruses as described herein.

### Retroviruses

Retroviruses are a genus of virus that insert a copy of their RNA genome into the DNA of host cells they invade. Retroviruses express glycoproteins on the surface of their viral envelopes. Interactions between the glycoproteins and lectins on the surface of host cells, e.g., human cells, facilitate the entry of retroviruses into human cells. Nonlimiting examples of retroviruses that cause infections in humans and cause infections that may be treated by the methods of the present invention include human immunodeficiency virus, human T-lymphotropic virus type 1, and human T-lymphotropic virus type 2.

### Human Norovirus

Human norovirus is the most common cause of gastroenteritis and causes an infection with symptoms including but not limited to diarrhea, vomiting, stomach pain, fever, headaches, and dehydration. Norovirus expresses a sialic acid-binding viral lectin that plays a role in the entry of norovirus into host cells.

### Herpesvirus

Herpesviruses are a genus of DNA viruses that infect animals. Nonlimiting examples of herpesviruses that infect human include herpes simplex virus 1, herpes simplex virus 2, varicella zoster virus, human cytomegalovirus, Epstein-Barr virus, roseolovirus, pseudorabies virus, and Kaposi's sarcoma associated herpesvirus. The internalization of herpesviruses into host cells is mediating by the binding of viral envelope glycoproteins to host cell lectins.

### Coronavirus

Coronaviruses are a family of RNA viruses that infect animals. Nonlimiting examples of coronaviruses that infect humans include SARS-CoV-2, SARS-CoV-1, MERS-CoV, human coronavirus 229E, human coronavirus NL63, human coronavirus OC43, and human coronavirus HKU1. The viral envelope of coronaviruses contains at least one glycoprotein, which facilitates entry into host cells.

### SARS-CoV-2

SARS-CoV-2 is a single-stranded RNA virus that causes the disease COVID-19. Nonlimiting examples of symptoms of COVID-19 infections include fever, cough, headache, fatigue, breathing difficulties, nasal congestion and runny nose, sore throat, diarrhea, and loss of smell and taste. The majority of individuals who suffer from COVID infections experience mild or moderate symptoms. However, approximately 15% of individuals who become infected with SARS-CoV-2 experience severe symptoms. Nonlimiting examples of severe symptoms include dyspnea, hypoxia, respiratory failure, shock, multiorgan dysfunction, or death. A subset of patients who become infected with SARS-CoV-2 experience “long-haul infections” in which COVID-19 symptoms including but not limited to fatigue, headaches, shortness of breath, loss of smell, muscle weakness, low fever, and cognitive dysfunction continue for a period of time (e.g., days, weeks, months) following their diagnosis.

COVID-19 transmission is thought to occur mainly through respiratory route via SARS-CoV-2 virions that are contained in the respiratory droplets and/or aerosols of individuals infected with COVID-19. Transmission occurs when the respiratory droplets or aerosols enter the mouth, nose, or eyes of a second individual. Approximately 1,000 COVID virons are believed to be sufficient to initiate a new infection.

### Orthomyxovirus

Orthomyxoviruses are a family of RNA viruses that infect animals. Orthomyxoviruses express glycoproteins on their surface that bind to lectins on the surface of host cells. Orthomyxoviruses that infect humans include alphainfluenzavirus, betainfluenzavirus, deltainfluenzavirus, gammainfluenzavirus, isavirus, thogotovirus, and qauranjavirus. Certain orthomyxoviruses, including alphainfluenzavirus, express viral lectins on their surface that facilitate entry into host cells by interacting with carbohydrates on the host cell surface.

### Adenovirus

Adenoviruses are a family of viruses that cause viral infections in humans and include the species human adenovirus A, human adenovirus B, human adenovirus C, human adenovirus D, human adenovirus E, human adenovirus F, and human adenovirus G. Adenoviruses express glycoproteins on their surface that bind to lectins on the surface of host cells.

### Flavivirus

Flaviviruses are a genus of enveloped single stranded RNA viruses that cause viral infections in humans and include the species of West Nile virus, dengue virus, tick-born encephalitis virus, yellow fever virus, Zika virus, hepatitis C virus, Murray Valley encephalitis virus, Tick-borne encephalitis virus, Saint Louis encephalitis virus, or Japanese encephalitis virus. Flaviviruses express glycosylated envelope proteins that interacts with host cell lectins during host cell entry.

### Rotavirus

Rotaviruses are a genus of double stranded RNA viruses that cause viral infections in humans. Human rotavirus infections are most common in children, and rotaviruses are the most common cause of diarrheal disease in infants and young children. Rotaviruses include the species rotavirus A, rotavirus B, rotavirus C, rotavirus D, rotavirus F, rotavirus G, rotavirus H, rotavirus I, or rotavirus J. Rotaviruses express a spike protein, VP4, that is proteolytically cleaved into VP8* and VP5* prior to infection. VP8* has lectin domains, and the entry of rotaviruses into host cells is facilitated by interactions between VP8* and carbohydrates on the surface of host cells.

### Mononegavirales

Mononegavirales is an order of viruses that include the family of orthopneumoviruses, the Ebola virus, the human respiratory syncytial virus, the measles virus, the mumps virus, the Nipah virus and the rabies virus. Viruses in the order mononegavirales express glycoproteins, which facilitate their entry into host cells.

### Orthopneumovirus

Orthopneumoviruses are a genus of single stranded RNA viruses which have nonsegmented genomes. Orthopneumoviruses that infect humans include human metapneumovirus, human respiratory syncytial virus A2, and human respiratory syncytial virus B1. Orthopneumoviruses express attachment glycoproteins and fusion glycoproteins, which bind to lectins on the surface of host cells during infection.

### Rubella Virus

The rubella virus is a single stranded RNA virus that causes the disease rubella in humans. Rubella expresses two membrane spanning glycoproteins, E1 and E2, that interact with host cell lectins during viral entry into host cells.

### Interactions Between Lectin-Binding Carbohydrates and Viral Proteins

The following description of lectin-binding carbohydrate activity is provided without wishing to be bound by theory.

In some embodiments, the lectin-binding carbohydrates bind to portions of viral proteins that have similar structures to lectins. For example, the N-terminal domain (NTD) of the SARS-CoV-2 spike protein is essential for vial entry into human cells. Portions of the SARS-CoV-2 spike protein resemble human galectins, which contain a highly conserved carbohydrate-binding domain. In some embodiments, the lectin-binding carbohydrates bind to the NTD of the SARS-CoV-2 spike protein. In some embodiments, binding of lectin-binding carbohydrates to the NTD of the SARS-CoV-2 spike protein is deleterious to the ability of SARS-CoV-2 to enter a human cell.

In some embodiments, lectin-binding carbohydrates prevent entry of viruses into human cells by inhibiting lectins on the surface of the virus, e.g., by allosterically inhibiting lectins on the surface of the virus. In some embodiments, lectin-binding carbohydrates bind to lectins on the surface of human cells. In some embodiments, the binding of lectin-binding carbohydrates to galectins on the surface of human cells is deleterious to the ability of viruses to enter human cells. In some embodiments, lectin-binding carbohydrates bind to carbohydrates that are displayed on the surface of the virus, e.g., on the viral envelope. In some embodiments, lectin-binding carbohydrates bind to carbohydrates that are displayed on the surface of the host cell, e.g., to glycolipids. In some embodiments, the binding of lectin-binding carbohydrates to carbohydrates displayed on the surface of the virus or the host cell is deleterious to the ability of SARS-CoV-2 to enter human cells. In some embodiments, lectin-binding carbohydrates recruit elements of the immune system, (e.g., leukocytes) to the virus. In some embodiments, lectin-binding carbohydrates deactivate viruses, which are then eliminated by the liver.

In some embodiments, lectin-binding carbohydrates stimulate the immune response against viruses in a subject. One element of the immune response is production of immunoglobulin G (IgG). In some embodiments, administration of lectin-binding carbohydrates results in a higher IgG antibody titer in the subject relative to the IgG antibody titer observed in the absence of galactomannan administration.

### Dosages

The dosage of the composition used in the methods described herein can vary depending on many factors, e.g., the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The composition used in the methods described herein may be administered initially in a suitable dosage that may be adjusted as required, depending on the clinical response. In general, a suitable daily dose of a compound of the invention will be that amount of the compound that is the lowest dose effective to produce a therapeutic effect. Such an effective dose will generally depend upon the factors described above.

While the attending physician ultimately will decide the appropriate amount and dosage regiment, in some embodiments an effective amount may about 10 mg/m², about 20 mg/m², about 40 mg/m², about 80 mg/m², or about 160 mg/m². In some embodiments, an effective amount may be between about 10 mg/m² and about 160 mg/m², between about 20 mg/m² and about 100 mg/m², between about 30 mg/m² and about 50 mg/m², or between about 35 mg/m² and about 45 mg/m². In some embodiments, an effective amount may be 10 mg/m² to 160 mg/m², 20 mg/m² to 100 mg/m², 30 mg/m² to 50 mg/m², or 35 mg/m² to 45 mg/m².

### Pharmaceutical Compositions

The administration of lectin-binding carbohydrates may be by any suitable means that results in treatment of a viral infection. Lectin-binding carbohydrates may be contained in any appropriate amount in any suitable carrier substance and are generally present in an amount of 1-95% by weight of the total weight of the composition. The composition may be provided in a dosage form that is suitable for the sublingual, buccal, oral, parenteral (e.g., intravenously, intramuscularly), pulmonary, intranasal, transdermal, vaginal, or rectal administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, sprays, vapors, or aerosols. The pharmaceutical compositions may be formulated according to conventional pharmaceutical practice (see, e.g., Remington: The Science and Practice of Pharmacy, (23rd ed.) ed. A. Adejare., 2020, Academic Press, Philadelphia, PA).

In some embodiments, the lectin-binding carbohydrates are formulated into a solution for IV administration. In some embodiments, the lectin-binding carbohydrates are administered intravenously as a continuous infusion. In some embodiments, the lectin-binding carbohydrates are administered intravenously as a bolus. In some embodiments, the lectin-binding carbohydrates are formulated in a solution for intravenous administration at a concentration of about 0.1 mg/mL, about 0.5 mg/mL, about 1 mg/mL, about 2 mg/mL, about 4 mg/mL, or about 8 mg/mL. In some embodiments, the lectin-binding carbohydrates are formulated in a solution for intravenous administration at a concentration of about 0.1 mg/mL-about 8 mg/mL, e.g., about 0.5 mg/mL-about 4 mg/mL, or about 1 mg/ml-about 2 mg/mL.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the active compound within the body over an extended period of time; (ii) formulations that after a predetermined lag time create a substantially constant concentration of the active compound within the body over an extended period of time; and (iii) formulations that sustain active compound action during a predetermined time period by maintaining a relatively, constant, effective active compound level in the body with concomitant minimization of undesirable side effects associated with fluctuations in the plasma level of the active compound (sawtooth kinetic pattern).

Any one of a number of strategies can be pursued in order to obtain controlled release of the active compound in question. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the active compound in a controlled manner. Examples include single or multiple unit tablet or capsule compositions, oil solutions, suspensions, emulsions, microcapsules, microspheres, nanoparticles, patches, and liposomes. In some embodiments of the method of the present invention, the composition of lectin-binding carbohydrates that is administered includes about 1% to about 50% (wt/wt) or about 1% to about 25% (wt/wt) of a lectin-binding carbohydrate of high molecular weight. In some embodiments, the composition includes about 20% to about 80% (wt/wt) of a lectin-binding carbohydrate of low molecular weight. In some embodiments, the lectin-binding carbohydrate includes about 40% to about 60% (wt/wt) of an oligosaccharide and/or monosaccharide. In some embodiments, the lectin-binding carbohydrate of high molecular weight has a high viscosity. In some embodiments, the lectin-binding carbohydrate of low molecular weight has a high solubility. In some embodiments, the ratio of low molecular weight lectin-binding carbohydrate to high molecular weight lectin-binding carbohydrate may be about 2 to 1 (wt/wt), 20 to 1 (wt/wt), and up to about 100 to 1 (wt/wt), inclusive of all ranges and sub-ranges in between. In some embodiments, the compositions described above may optionally include one or more additional additives. In some embodiments, the lectin-binding carbohydrates are formulated with carbohydrates that do not bind to lectins in order to improve the physical properties of the composition (e.g., the viscosity or solubility of the composition). In an illustrative embodiment, an additional additive may include one or more sugar alcohols, including, but not limited to, sorbitol, erithritol, inositol, and other sugar alcohols of the type. A non-limiting list of other potential additional additives includes vitamins and minerals at their recommended % daily value requirements.

### Solid Dosage Forms for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These compositions can be prepared in a variety of ways well known in the pharmaceutical art and can be made so as to release the lectin-binding carbohydrates in specific segments of the gastrointestinal tract at controlled times by a variety of excipients and formulation technologies. For example, formulations may be tailored to address a specific disease, to achieve plasma levels of the lectin-binding carbohydrates required to achieve therapeutic efficacy, to enable a desired duration of drug effect, and to provide a set of compositions with varying drug release.

The oral dosage forms contemplated by the invention may include the lectin-binding carbohydrates in a mixture with non-toxic pharmaceutically acceptable excipients. Pharmaceutically acceptable excipients are known to the skilled artisan. Excipients may be, for example, inert diluents or fillers such as sucrose, sorbitol, sugar, mannitol, microcrystalline cellulose, in particular microcrystalline cellulose PH101 or microcrystalline cellulose PH200, starches including potato starch, calcium carbonate, sodium chloride, lactose, calcium phosphate, calcium sulfate, or sodium phosphate; disintegrants such as crospovidone, sodium alginate, colloidal magnesium aluminum silicate, calcium silicate, sodium starch glycolate, acrylic acid derivatives, microcrystalline cellulose, sodium carboxymethyl cellulose, calcium carboxymethyl cellulose, modified cellulose gum, cross-linked povidone, alginic acid and alginates, pregelatinised starch, modified corn starch cellulose derivatives including microcrystalline cellulose, starches including potato starch, croscarmellose sodium, alginates, or alginic acid; binders such as sucrose, glucose, sorbitol, acacia, alginic acid, gelatin, starch, pregelatinized starch, microcrystalline cellulose, magnesium aluminum silicate, carboxymethylcellulose sodium, methylcellulose, hydroxypropyl methylcellulose, hydroxypropyl cellulose EXF, ethylcellulose, polyvinylpyrrolidone, or polyethylene glycol; lubricants and/or glidants such as colloidal silicon dioxide, particularly colloidal silicon dioxide Cab-O-Sil® M5P, glycerol tribehenate, magnesium stearate, calcium stearate, talc, sodium stearyl fumarate, sodium behenate, stearic acid, cetyl alcohol, polyoxyethylene glycol, leucine, sodium benzoate, stearates, polyethylene glycol, glyceryl monostearate, glyceryl palmitostearate, liquid paraffin, poloxamer, sodium lauryl sulphate, magnesium lauryl sulphate, hydrogenated castor colloidal silicon dioxide, palmitostearate, stearic acid, zinc stearate, stearyl alcohol, silicas, or hydrogenated vegetable oil; anti-caking agents such as colloidal silicon dioxide, microcrystalline cellulose, tricalcium phosphate, microcrystalline cellulose, magnesium stearate, sodium bicarbonate, sodium ferrocyanide, potassium ferrocyanide, calcium ferrocyanide, calcium phosphate, sodium silicate, colloidal silicon dioxide, calcium silicate, magnesium trisilicate, talcum powder, sodium aluminosilicate, potassium aluminum silicate, calcium aluminosilicate, bentonite, aluminum silicate, stearic acid, polydimethylsiloxane. Other pharmaceutically acceptable excipients may be colorants, flavoring agents, plasticizers, humectants, and buffering agents.

Suitable pharmaceutical carriers, as well as pharmaceutical necessities for use in pharmaceutical formulations, are described in Remington: Remington: The Science and Practice of Pharmacy, (23rd ed.) ed. A. Adejare., 2020, Academic Press, Philadelphia, PA, and in the USP44/NF39 (United States Pharmacopeia and the National Formulary) or corresponding European or Japanese reference documents.

The solid compositions of the invention may include a coating adapted to protect the composition from unwanted chemical changes, (e.g., chemical degradation prior to the release of the active substances). The coating may be applied on the solid dosage form in a similar manner as that described in Encyclopedia of Pharmaceutical Technology (4th ed.) ed. J. Swarbrick, 2013, CRC Press, Boca Raton, FL.

Powders and granulates may be prepared using the ingredients mentioned above in a conventional manner using, e.g., a mixer, a fluid bed apparatus, melt congeal apparatus, rotor granulator, extrusion/spheronizer, or spray drying equipment.

In some embodiments, the pharmaceutical composition of the lectin-binding carbohydrates is formulated in a solid oral dosage form. In some embodiments, the solid oral dosage form of the lectin-binding carbohydrates is intended to be dissolved in the mouth of the subject. In some embodiments, the solid oral dosage form is chewable, e.g., a chewable tablet. In some embodiments, the subject chews the solid oral dosage form and holds the solid oral dosage form of the lectin-binding carbohydrates in their mouth for at least 1 minute, e.g., at least 2 minutes, at least 3 minutes, at least 4 minutes, at least 5 minutes prior to swallowing. In some embodiments, the subject does not chew the solid oral dosage form and holds the pharmaceutical composition of the lectin-binding carbohydrates in their mouth for at least 1 minute, e.g., at least 2 minutes, at least 3 minutes, at least 4 minutes, at least 5 minutes prior to swallowing. In some embodiments, the subject holds the pharmaceutical composition of the lectin-binding carbohydrates in their mouth until it is substantially dissolved. In some embodiments, the subject holds the composition of the lectin-binding carbohydrates in their mouth until the lectin-binding carbohydrates have contacted at least 50% (at least 50%, at least 60%, at least 70%, at least 80%, at least 90%, at least 95%), of the area of their oral mucosa.

### Methods of Treatment

Lectin-binding carbohydrates, or pharmaceutical compositions thereof, may serve as a useful therapeutic for viral infections. In particular, pectin or galactomannan polysaccharides may be useful in treating the symptoms of a viral infection in a subject. In some embodiments, the subject is an adult (e.g., the subject is greater than 18 years old). In some embodiments, the subject is a child (e.g. the subject is less than 18 years old, less than 17 years old, less than 16 years old, less than 15 years old, less than 14 years old, less than 13 years old, less than 12 years old, less than 11 years old, less than 10 years old, less than 9 years old, less than 8 years old, less than 7 years old, less than 6 years old, less than 5 years old, less than 4 years old, less than 3 years old, less than 2 years old, less than 1 year old).

In some embodiments, the lectin-binding carbohydrates are administered fewer than 48 hours following the diagnosis of a viral infection in the subject (e.g., fewer than 24 hours following the diagnosis of a viral infection in the subject, fewer than 12 hours following the diagnosis of a viral infection in the subject, less than 6 hours following the diagnosis of a viral infection in the subject, less than 3 hours following the diagnosis of a viral infection in the subject, at substantially the same time as a viral infection is diagnosed in the subject).

In some embodiments, including any of the foregoing embodiments, the lectin-binding carbohydrates are administered more than 30 minutes after the subject consumes food, e.g., more than 60 minutes, more than 90 minutes, or more than 120 minutes after the subject consumes food.

In some embodiments, the lectin-binding carbohydrates are administered to the subject at least once per day. In some embodiments, including any of the foregoing embodiments, the lectin-binding carbohydrates are administered to the subject at least twice per day. In some embodiments, including any of the foregoing embodiments, the lectin-binding carbohydrates are administered to the subject at least three times per day. In some embodiments, including any of the foregoing embodiments, the lectin-binding carbohydrates are administered to the subject at least four times per day. In some embodiments, including any of the foregoing embodiments, the lectin-binding carbohydrates are administered to the subject at least five times per day. In some embodiments, including any of the foregoing embodiments, the lectin-binding carbohydrates are administered to the subject at least six times per day. In some embodiments, including any of the foregoing embodiments, the lectin-binding carbohydrates are administered to the subject at least seven times per day. In some embodiments, including any of the foregoing embodiments, the lectin-binding carbohydrates are administered to the subject at least eight times per day. In some embodiments, including any of the foregoing embodiments, the lectin-binding carbohydrates are administered to the subject at least nine times per day. In some embodiments, including any of the foregoing embodiments, the lectin-binding carbohydrates are administered to the subject at least ten times per day. In some embodiments, including any of the foregoing embodiments, the lectin-binding carbohydrates are administered to the subject at least eleven times per day. In some embodiments, including any of the foregoing embodiments, the lectin-binding carbohydrates are administered to the subject at least twelve times per day. In some embodiments, including any of the forgoing embodiments, the method comprises administering to the subject the lectin-binding carbohydrates hourly, e.g., during waking hours.

### EXAMPLES

### Example 1: Antiviral Testing-Sars-CoV-2

### Anti-Viral Assay

To determine anti-viral effects from ProLectin-M and ProLectin-I against SARS CoV-2, Vero cells were first treated with various concentrations of each compound. After 2 hours, the compounds in the culture medium were removed, and viral stock (˜multiplicity of infection (MOI) 0.1 in DMEM culture media without FBS) was used to infect Vero cells at 37° C. for 3 hours. The unabsorbed virus in the culture medium was then removed, and cells were washed and overlaid with 1 mL of fresh DMEM containing 10% FBS and test compounds. After 48 hours, the viral supernatant was collected, and qRT-PCR was used to determine the reduction in viral RNA copy number as previously described. Uninfected Vero cells and those infected with viral stock were used as cell and infection controls, respectively.

For these experiments, two protocols were used. In Protocol 1, Vero cells on the plate were initially treated with ProLectin-M or ProLectin-I prior to being infected with Sars-Cov-1 (DMSO was used as the control). In Protocol 2, Vero cells were initially cultured with the Sars-CoV-2 virus prior to being treated with drug-spiked media. FBS was used as the control.

Cells released from and bound to SARS-CoV-2 virus were analyzed to calculate the half-maximal effective concentration (EC50) and half-inhibitory concentration (IC50) values of our test compounds by plotting % viral reduction or viral presence vs. log concentrations of the test compounds.

### Drug Treatment

Vero cells were cultured in 96-well plates at 37° C. with 5% CO₂ in DMEM supplemented media with 10% (v/v) FBS and 3.7 g/L sodium bicarbonate. At 90-95% confluency, cells were primed with complete medium containing different concentrations of either ProLectin M and ProLectin I for 2 hours. The wells containing the test compounds were replaced with the virus (˜MOI 0.1) in DMEM culture media (without FBS) for 3 hours. Later, the virus-containing medium was aspirated and replaced with fresh DMEM containing 10% FBS and test compounds. Culture supernatant was collected for real time-PCR analysis of viral RNA copy.

### Viral RNA Extraction

Viral RNA was extracted from 200 μL aliquots of culture supernatants using the MagMAX™ viral/pathogen extraction kit (Applied Biosystems, Thermo Scientific). The viral supernatants from the test groups were mixed with a lysis buffer containing 260 μL of MagMAX™ viral/pathogen binding solution, 10 μL of MVP II binding beads, and 5 μL of MagMAX™ viral/pathogen proteinase-K for a total of 200 μL of sample in a deep well plate (KingFisher™, Thermo Scientific). RNA extraction was performed using KingFisher Flex system (version 1.01, Thermo Scientific) by following manufactures instructions. The eluted RNA was stored at −80 C until further use.

### Rt-qPCR for Detection of SARS CoV-2

Quantitative PCR was performed using a Meril Covid-19 one step Real-Time PCR kit to detect the ORF1ab (FAM labeled) and nucleoprotein N (HEX labeled) genes of SARS-CoV-2 in the isolated RNA samples. Reaction conditions were set up according to manufacturer's protocol: 15 min at 50° C. (reverse transcription), 3 min at 95° C. (cDNA initial denaturation) followed by 15 sec at 95° C. (45 cycles of denaturation), 40 sec at 55° C. (annealing, extension and fluorescence measurement), and 10 sec at −25° C. (cooling). The program was set up using QuantStudio-5 machine (Thermo fisher). The threshold cycles (Ct) values of N gene (gene specific to SARS-CoV-2) were considered to plot the graphs.

### Statistical Analysis

Plaque forming unit (PFU) determined stock titres (ranging from 10⁴-10⁵) were used to develop a regression equation and calculate the percentage of viral reduction. After 72 hours of infection, the viral inhibition effect of both compounds was tested. The end point was calculated as a percentage of viral reduction using the following formula:

$$\% Viral Reduction = \frac{\begin{matrix}number of viral particles in infection \\ control - number of viral particles exposed\end{matrix}}{number of viral particles in infection control}$$

The experiment was repeated twice, and the results were averaged to calculate the % viral reduction. The regression equation for viral particles vs. Ct value of the N-gene specific to SARS-CoV-2 virus (y=3.5422x+40.786; R²=0.99, x=viral particle number, and y=Ct value).

The number of viral particles calculated as

$$X = ( 40.786 - Ct Rd / Rp - genes at different time points ) / 3.5422 .$$

Any log reduction in the % viral particles in the experiments was interpreted as an efficacy of the test compounds in blocking viral infectivity to the Vero cells. The student t-test was used to compare the data to the control, and all data are presented as the mean±SD.

### Results

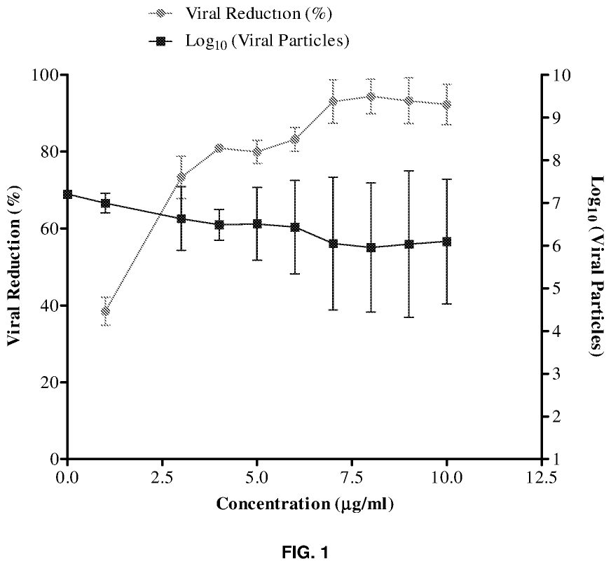

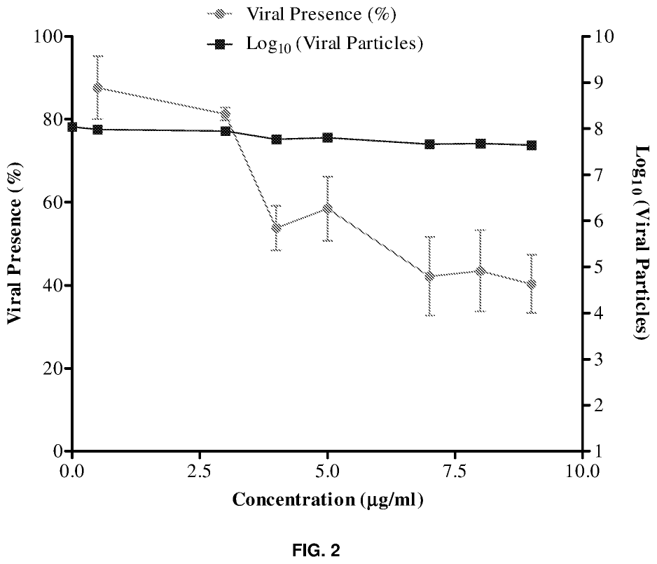

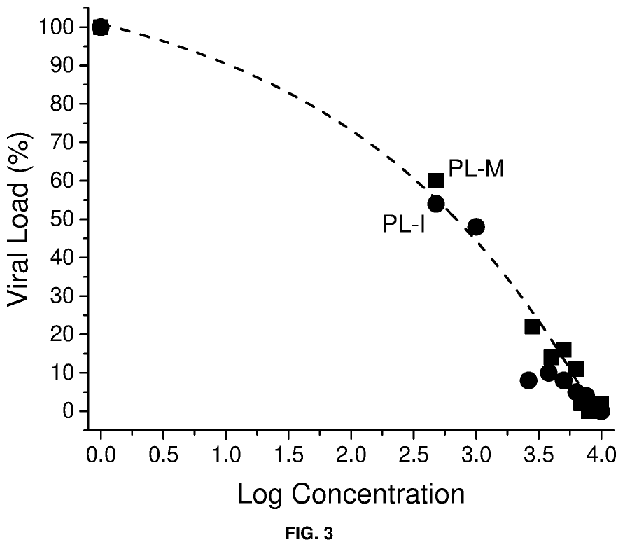

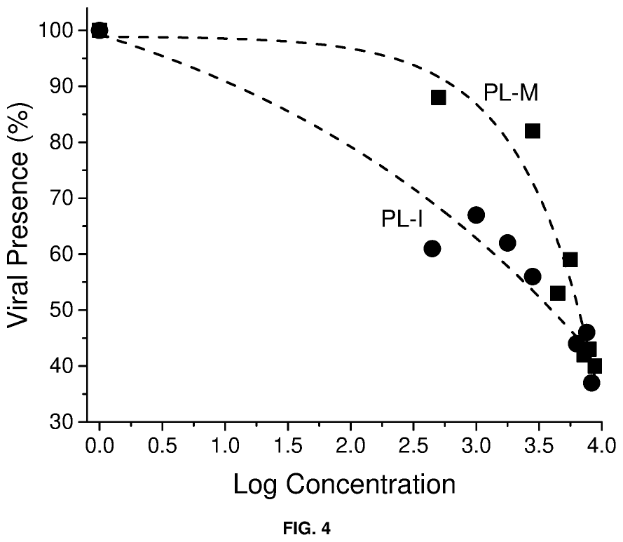

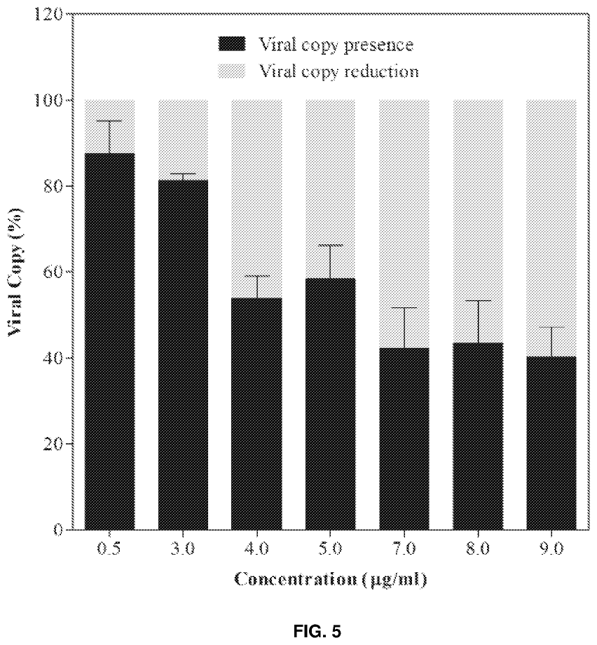

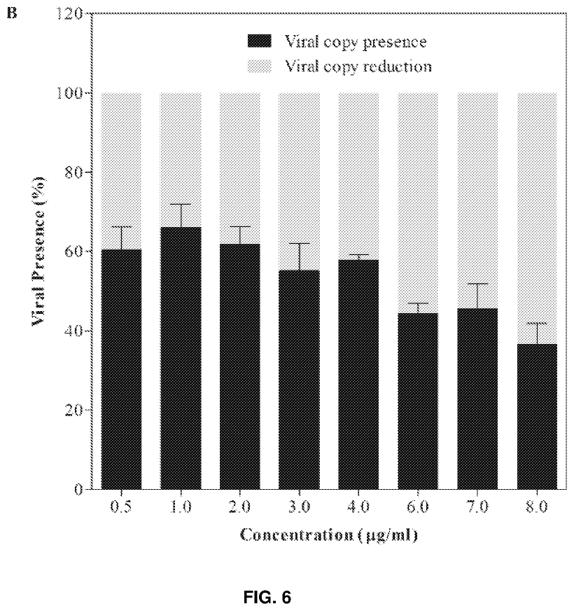

Anti-viral assays (protocols 1 and 2 for pre- and post-treatment, respectively) with SARS-CoV-2 showed that both ProLectin-M (PL-M) and ProLectin-I (PL-I) render a nearly 99 percent (i.e. 2 log) reduction in viral RNA copy number compared to control (FIGS. 1-6, Table 1). Pre-treatment (protocol 1) and post-treatment (protocol 2) of Vero cells with PL-M reduced viral load >90%, i.e., from 7 g/ml to 10 μg/ml, with the greatest reductions being observed before (94.37%) and after (59.68%) treatment with PL-M at doses of 8 μg/ml and 9 μg/ml, respectively. Viral particles were reduced from 10^{6.9} to 10^{5.1} and from 10^{6.9} to 10^{5.5} before and after PL-M treatment, respectively (FIGS. 1-6).

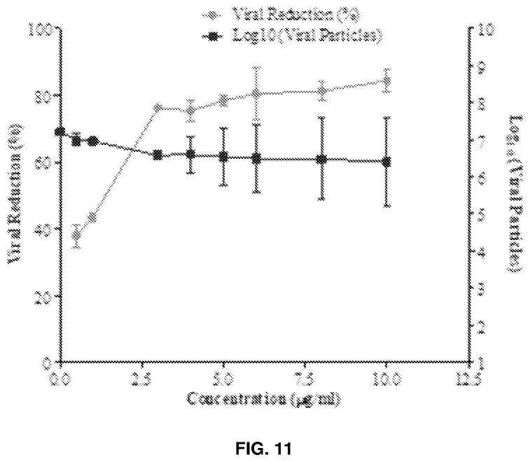

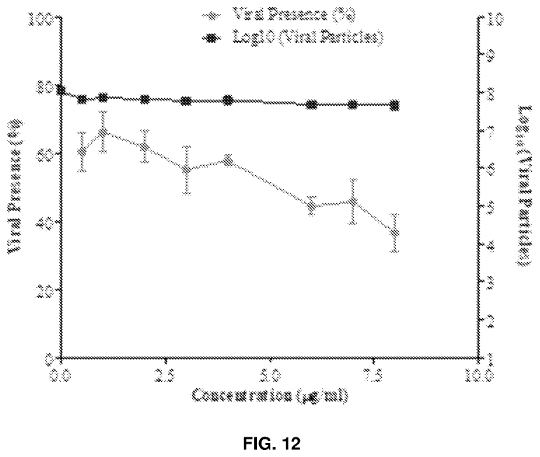

Treatment with PL-I also demonstrated a significant reduction in viral load (Table 1, FIGS. 11 and 12), with viral particles being decreased from 10^{6.8} to 10^{5.1} (before) and from 10^{6.8} to 10^{5.2} (after). Based on viral load reduction, IC50 values for PL-M and PL-I were 6.18 μg/ml and 4.25 μg/ml, respectively, with EC50 values being 1.53 g/ml and 0.87 μg/ml, respectively.

TABLE 1
Anti-Sars-CoV-2 effects of PL-M and PL-I in pre-treated
(Protocol 1) and post-treated (Protocol 2) Vero cells.

| | | | ProLectin M | | ProLectin I |
|---|---|---|---|---|---|
| | | Treatment | Viral load | Treatment | Viral load |
| Protocol | Experimental | (μg/ml) | reduction (%) | (μg/ml) | reduction (%) |
| Protocol 1 | Vero cells on a plate | 1.0 | 38.41 ± 3.62 | 0.5 | 37.85 ± 3.46 |
| | first treated with the | 3.0 | 73.33 ± 5.52 | 1.0 | 43.43 ± 1.45 |
| | drug before being | 4.0 | 80.92 ± 1.01 | 3.0 | 76.21 ± 0.55 |
| | infected with SarsCov- | 5.0 | 79.94 ± 2.99 | 4.0 | 75.31 ± 3.09 |
| | 2/control (DMSO) | 6.0 | 83.16 ± 3.11 | 5.0 | 78.41 ± 1.78 |
| | | 7.0 | 93.03 ± 5.69 | 6.0 | 80.43 ± 7.85 |
| | | 8.0 | 94.37 ± 4.50 | 8.0 | 81.27 ± 2.75 |
| | | 9.0 | 93.26 ± 5.96 | 10.0 | 84.31 ± 3.33 |
| | | 10.0 | 92.26 ± 5.23 | | |
| | | | Viral presence | | Viral presence |
| | | | (%) | | (%) |
| Protocol 2 | Vero cells cultured | 0.5 | 87.63 ± 7.56 | 0.5 | 60.46 ± 5.72 |
| | with the Sars-CoV-2 | 3.0 | 81.30 ± 1.52 | 1.0 | 66.09 ± 5.88 |
| | virus before being | 4.0 | 53.78 ± 5.32 | 2.0 | 61.79 ± 4.61 |
| | treated with drug- | 5.0 | 58.45 ± 7.71 | 3.0 | 55.15 ± 6.90 |
| | spiked medium/ | 7.0 | 42.19 ± 9.53 | 4.0 | 57.78 ± 1.40 |
| | control medium (FBS) | 8.0 | 43.48 ± 9.78 | 6.0 | 44.47 ± 2.63 |
| | | 9.0 | 40.32 ± 7.0 | 7.0 | 45.67 ± 6.26 |
| | | | | 8.0 | 36.59 ± 5.34 |

Values represent mean ± SD from triplicate analysis.

### Cytotoxicity Assay

Cell viability was determined using the MTT [3-(4,5-dimethylthiazol-2-yl)-2,5-diphenyltetrazolium bromide; Sigma-Aldrich] assay. Vero cells were plated in triplicate in 96 well culture plates and incubated at 37° C. with 5% CO₂. After reaching 90-95% cell confluency, different concentrations of ProLectin-M and ProLectin-I were added to the cells for 24 hours to assess the cytotoxic effect of the compounds on the cells. After 24 hours, 100 μl of MTT substrate (final concentration 50 μg/ml) was added to the cells, and the plate was incubated for 3 hours at 37° C. with 5% CO₂. Later, the formed formazan crystals were dissolved in 100 μl of DMSO, and a multimode microplate reader, Synergy HI (Agilent Technologies Inc., USA), was used to measure absorbance at 570 nm, and the percentage of cell viability was calculated.

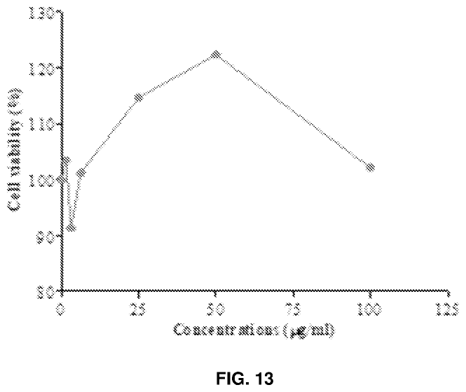

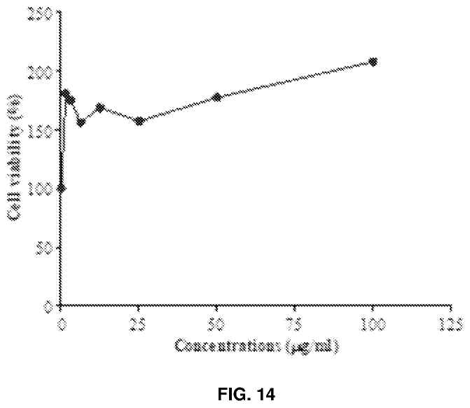

The MTT assay demonstrated that neither PL-M nor PL-I exhibited cytotoxic effects on Vero cells at concentrations up to 100 μg/ml, with CC50 values being >100 μg/ml (FIGS. 13 and 14). In fact, both compounds appeared to increase cell viability, with maximal effects from PL-M and PL-I on cell proliferation observed at doses of 50 μg/ml (122.31±0.10%) and 100 μg/ml (208.08±0.27%), respectively, when compared to control (100%).

### Example 2: Binding of Prolectin-M to Galectin 3

We hypothesized that PL-M functions in situ by binding to and antagonizing Gal-3 that normally interacts with SARS-CoV-2 to promote viral entry into cells. To validate this proposal, we used NMR spectroscopy to assess interactions between PL-M and the lectin.

Uniformly ¹⁵N-labeled galectin-3 (Gal-3) was dissolved at a concentration of 20 μM in 20 mM potassium phosphate buffer at pH 6.9, made up using a 95% H₂O/5% D₂O mixture. ¹H-¹⁵N HSQC NMR experiments were used to investigate binding of PL-M. ¹H and ¹⁵N resonance assignments for recombinant human Gal-3 were previously reported (Ippel et al., Biomol. NMR Assign. 9, 59-63 (2015)).

NMR experiments were carried out at 30° C. on a Bruker 850 MHz spectrometer equipped with a H/C/N triple-resonance probe and an x/y/z triple-axis pulse field gradient unit. A gradient sensitivity-enhanced version of two-dimensional ¹H-¹⁵N HSQC was applied with 256 (t1)×2048 (t2) complex data points in nitrogen and proton dimensions, respectively. Raw data were converted and processed by using NMRPipe (Delaglio et al., J Biomol NMR 6, 277-293 (1995)) and were analyzed by using NMRview (Johnson & Blevins, J Biomol NMR 4, 603-614 (1994).

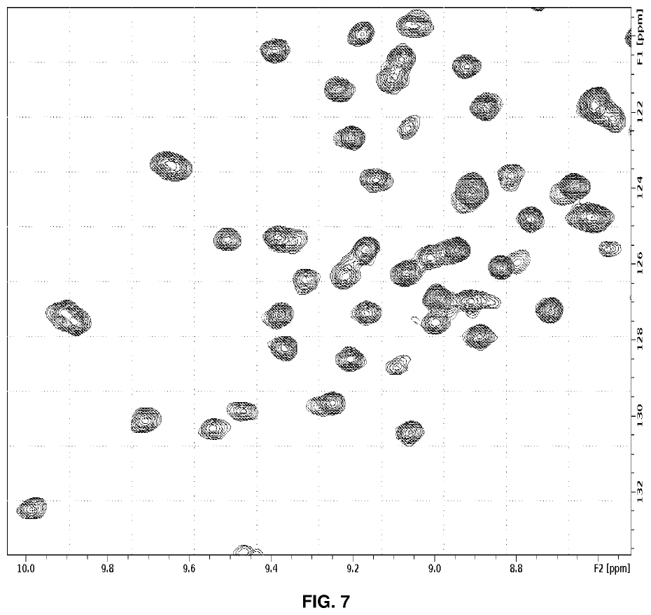

HSQC NMR spectra of ¹⁵N-labeled Gal-3 (¹⁵N-Gal-3) were measured as a function of PL-M concentration (0.3, 0.6, 1.2, 2.4 and 4.8 mg/mL). An ¹⁵N-1H HSQC spectral expansion is shown in FIG. 7 for ¹⁵N-Gal-3 in the absence (peaks in black) and presence (peaks in red) of 1.2 mg/ml PL-M. During the titration, Gal-3 resonances were differentially chemically shifted and reduced in intensity (broadened), with some peaks being so broadened by the end of the titration that they fell into the noise. This observation alone demonstrated that Gal-3 indeed binds to PI-M and indicates that the overall structure of Gal-3 was not significantly perturbed by the binding interactions. Moreover, the fact that resonances are generally significantly broadened and minimally chemically shifted, PL-M binding to Gal-3 fell in the intermediate exchange regime on the chemical shift time scale, suggesting that the equilibrium dissociation constant (K_{D}) lies in the 2 μM to 100 μM range. The extent of resonance broadening is related to binding affinity and stoichiometry, in addition to any binding-induced changes in internal motions and conformational exchange. Therefore, one might look at this as binding avidity, or the net ability of PL-M to bind to Gal-3. Alternatively, the apparent broadening could have resulted from multiple binding modes given the heterogeneous nature of the PL-M polysaccharide. Either way, Gal-3 bound to PI-M.

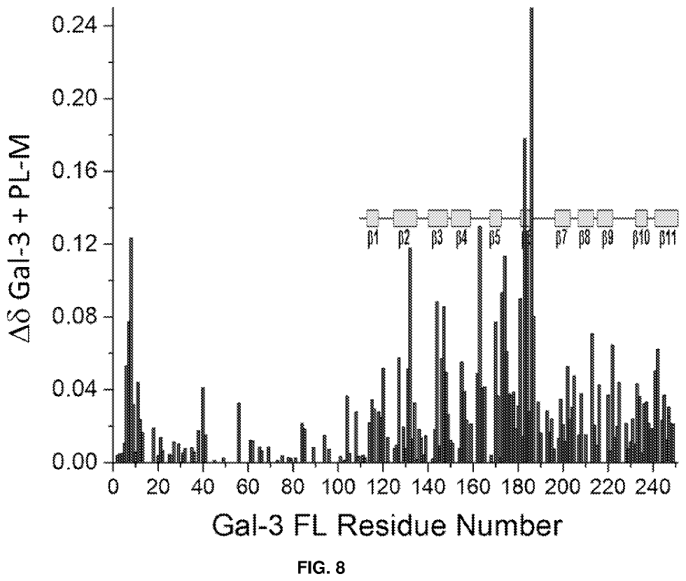

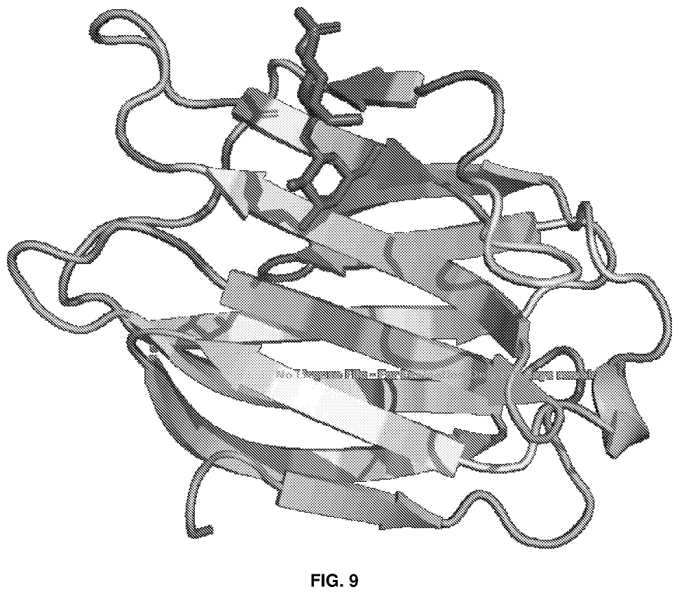

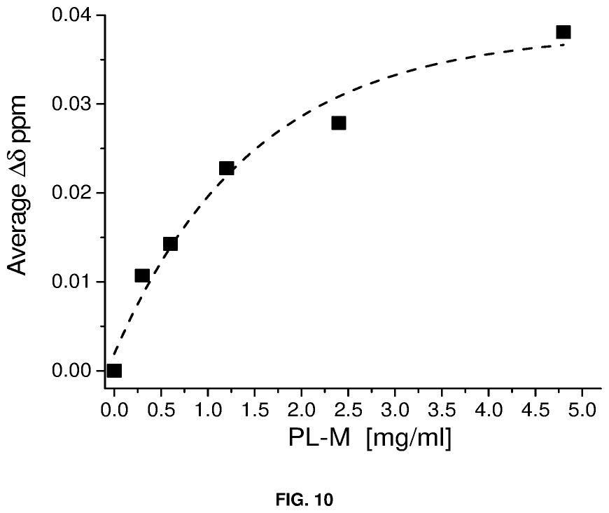

Even though PL-M binding-induced ¹⁵N-Gal-3 chemical shift changes, Δδ, were relatively small, they were useful to assess the location of the binding epitope on the lectin. FIG. 8 plots ¹⁵N-Gal-3 chemical shift changes, Δδ, vs. the amino acid sequence of Gal-3. The most shifted resonances arose from Gal-3 CRD residues in β-strands 3, 4, 5 and 6 that comprise the S-face βsheet of the β-sandwich to which the β-galactoside lactose binds, as illustrated in FIG. 9 that shows the structure of the Gal-3 CRD (pdb access code: 1A3K) with the most shifted residues being color highlighted. This indicated that the PL-M binding epitope on Gal-3 was within the canonical sugar binding domain on the S-face of the CRD. Chemical shift changes within the N-terminal tail (NT) (residues 1-111) most likely resulted from PL-M binding-induced allosteric effects on the CRD F-face and modulation of interaction dynamics between the NT and CRD F-face (Ippel et al., Glycobiology 26, 888-903 (2016)). FIG. 10 plots chemical shift changes averaged over all Gal-3 resonances vs. the concentration of PL-M with the 50% saturation point in the plot falling at ˜1 mg/ml. However, because Gal-3 binding fell in the intermediate exchange regime, one cannot accurately determine binding affinity (or stoichiometry), other than to say that the equilibrium dissociation constant, K_{d}, falls in the ˜2 μM to ˜100 μM range (Williamson, Prog. Nucl. Magn. Reson 73, 1-16 (2013)).

### Example 3: NMR Analysis of Prolectin-M

### Glycosyl Linkage Analysis

A Prolectin-M tablet was suspended in 50 mL of nanopure water. The supernatant of the suspension was lyophilized. Aliquots of 1.0 mg of Prolectin-M were used for linkage analysis. The sample was stirred in 400 μL of anhydrous dimethyl sulfoxide (DMSO) for 2 days until the samples were dissolved. Permethylation was achieved by two rounds of treatment with sodium hydroxide (NaOH) base (30 min) and iodomethane (90 min). The sodium hydroxide base was prepared according to the protocol described by Anumula and Taylor (1992) Anal. Biochem. 203:101-108. Briefly, to 100 μL of 50% w/w NaOH was added 200 μL of methanol (MeOH), and the mixture was vortexed. Then 2 mL of DMSO was added, and the base solution was vortexed and centrifuged repeatedly up to 5 times to remove residual sodium carbonate. After the final extraction, 2 mL of DMSO was added to the NaOH pellet and the solution was vortexed. Of this final base solution, 400 μL was added to the sample, and the mixture was sonicated for 30 min. Then, 100 μL of iodomethane was added, and the sample was stirred magnetically at room temperature for 45 min. A second round of base and then iodomethane was performed to ensure complete methylation.

The permethylated materials were hydrolyzed with 2 M TFA for 2 h at 121° C. and dried down with isopropanol under a stream of nitrogen. The samples were then reduced with 10 mg/mL NaBD₄ in 100 mM NH₄OH overnight, neutralized with glacial acetic acid, and dried with methanol. Finally, the sample was O-acetylated using 250 μL of acetic anhydride and 250 μL of concentrated trifluoroacetic acid (TFA) at 45° C. The sample was dried under N₂ stream, reconstituted in dichloromethane, and washed with nanopure water before injection into GC-MS (Table 2).

The resulting partially methylated alditol acetates (PMAAs) were analyzed on an Agilent 7890A GC interfaced to a 5975C MSD; separation was performed on a Supelco 2331 fused silica capillary column (30 m×0.25 mm ID) with a temperature gradient detailed in Table 2. The method was a derivation of the linkage method detailed by Heiss et al. (2009) Carbohydr. Res. 344:915-920.

TABLE 2

| | Rate (° C./min) | Value (° C.) | Hold Time (min) |
|---|---|---|---|
| Initial | | 60 | 1 |
| Ramp1 | 27.5 | 170 | 0 |
| Ramp2 | 4 | 235 | 2 |
| Ramp3 | 3 | 240 | 12 |

### NMR Analysis:

Sample Prolectin-M 9.2 mg was weighed and resuspended in 600 μl of D₂O. The supernatant was transferred into an NMR tube. Ten microliters of 1 mM DSS was added into the NMR tube as internal standard. The sample was analyzed at 34° C. (to shift the water signal away from the anomeric signals of Man) with a Bruker 900 MHZ NMR instrument equipped with a cryoprobe. A standard zgf2pr pulse sequence was employed with a pre-saturation sequence for water suppression. Pulse sequences, hsqcetgpsisp2, clmlevphpr, and noesygpphf2pr, were applied for collecting HSQC, TOCSY, and NOESY spectrum, respectively.

### Results

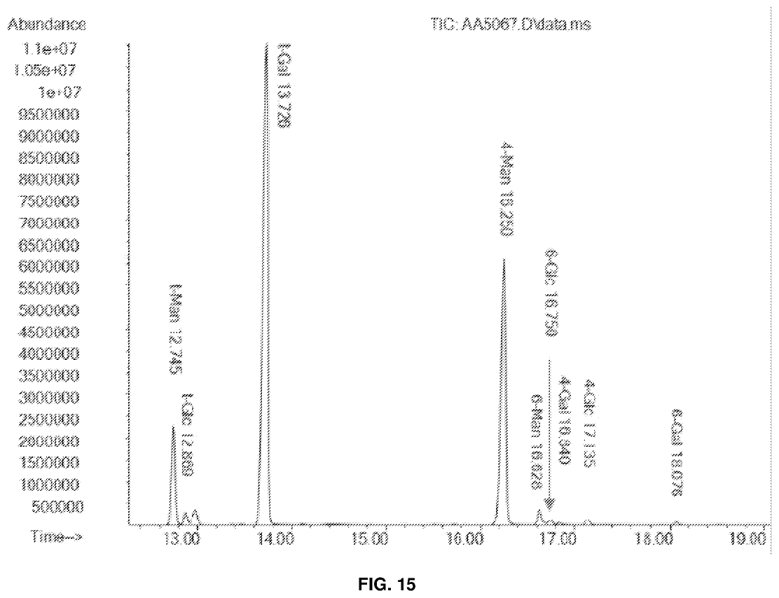

The glycosyl linkage analysis chromatogram is shown in FIG. 15, and the results are listed in Table 3. In summary, the most abundant linkages of Prolectin-M were 4-Manp, 4,6-Manp, and t-Galp, suggesting a galactomannan structure. The ratio of t-Galp to Manp of the sample was about equal to the ratio of branching Manp to all Manp, or about 2:3. This indicated that the mannan backbone is glycosylated with about 2 Gal on every 3 Man residues. The ratio of terminal Manp to internal Manp was about 1:10, suggesting an average back bone chain length of 11 Man residues. Together with the side chain Galp residues, this resulted in an average molecular weight of about 3 kDa.

TABLE 3
Relative percentage of each detected linkage in Prolectin-M.

| | Relative Area |
|---|---|
| Glycosyl Linkage Residue | Percentage (%) |
| Terminal Mannopyranosyl Residue (t-Manp) | 5.7 |
| Terminal Glucopyranosyl Residue (t-Glcp) | 0.8 |
| Terminal Galactopyranosyl Residue (t-Galp) | 36.5 |
| 4-linked Mannopyranosyl Residue (4-Manp) | 19.4 |
| 6-linked Mannopyranosyl Residue (6-Manp) | 1.1 |
| 6-linked Glucopyranosyl Residue (6-Glcp) | 0.4 |
| 4-linked Galactopyranosyl Residue (4-Galp) | 0.2 |
| 4-linked Glucopyranosyl Residue (4-Glcp) | 0.4 |
| 6-linked Galactopyranosyl Residue (6-Galp) | 0.3 |
| 4,6-linked Mannopyranosyl Residue (4,6-Manp) | 35.0 |
| 4,6-linked Glucopyranosyl Residue (4,6-Glcp) | 0.3 |
| Total | 100.0 |

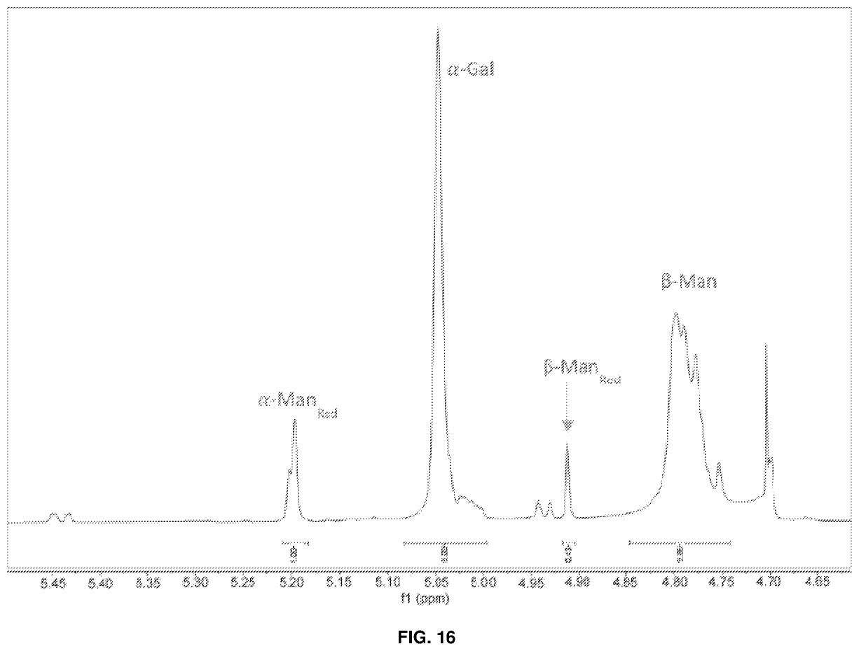

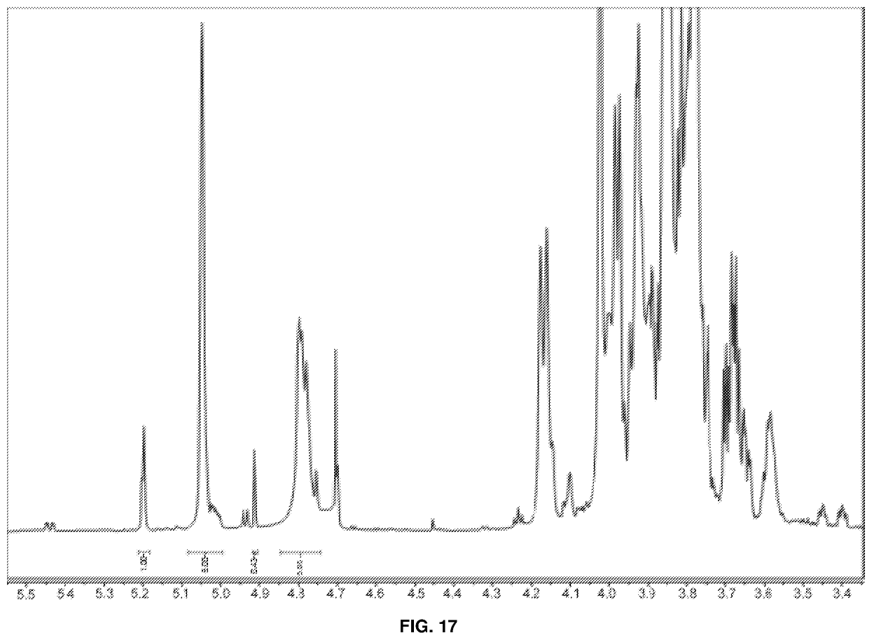

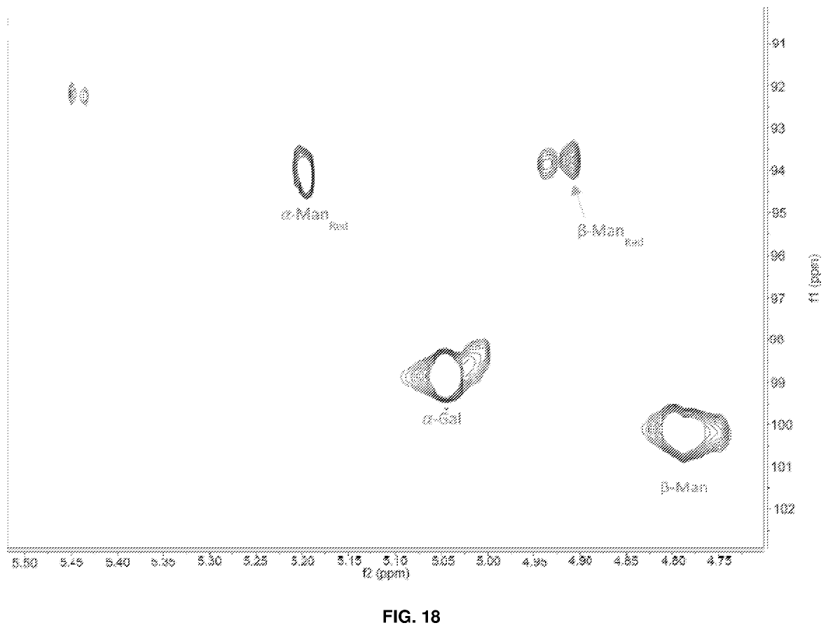

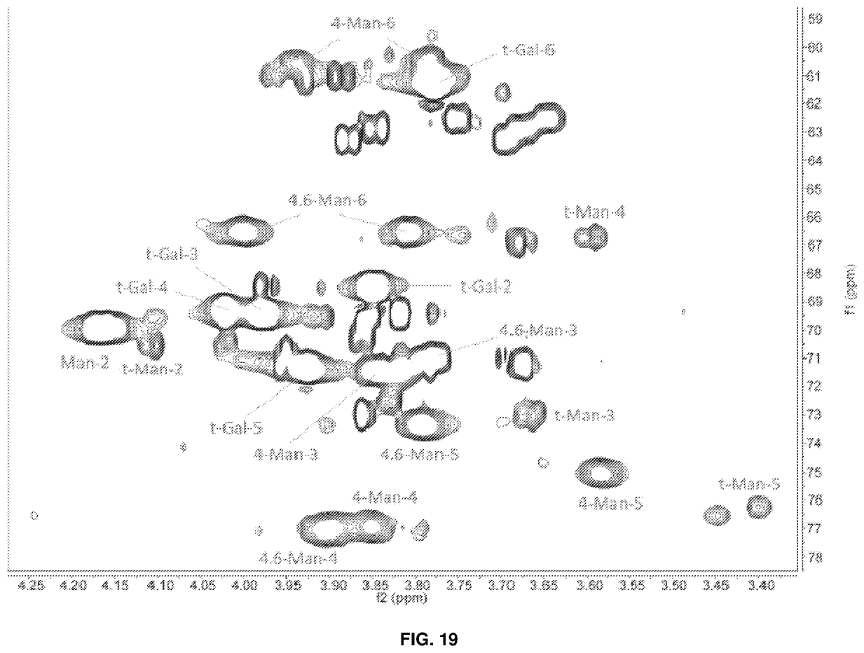

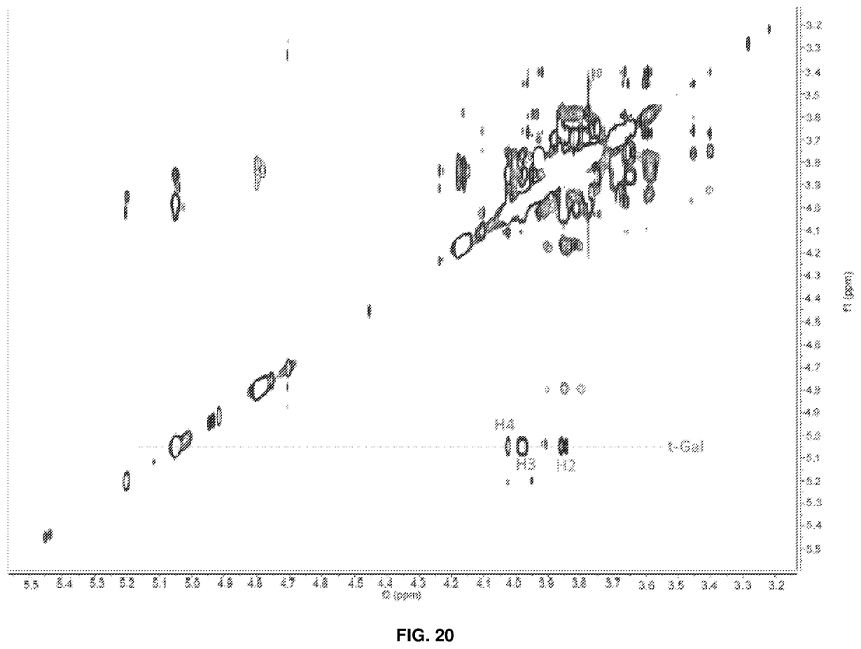

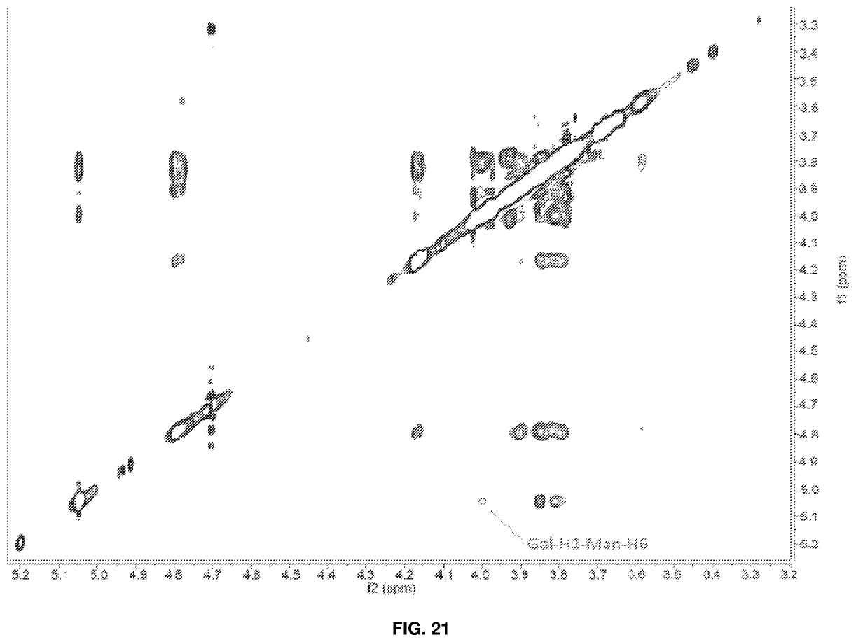

The NMR spectra are shown in FIGS. 16-21. The integration ratio between α-Gal H1 (8.00) and Man H1 [including reducing end Man (1.43 for both a and B configurations) and inner Man (9.86)] in the ¹H-NMR spectrum was about 1:1.4, suggesting the mannan backbone is glycosylated with about 2 Gal on every 3 Man residues, in agreement with the results from the glycosyl linkage analysis. The ¹H/¹³C cross-peaks are assigned and labeled in FIG. 18 and FIG. 19. The chemical shifts of these major glycosyl residues are summarized in Table 8. The assignment of α-Gal signals was based on a TOCSY spectrum (FIG. 20), while the assignment of backbone Man cross-peaks was based on literature (see references below). A NOESY spectrum confirmed the linkage between t-Gal and backbone Man by NOEs between α-Gal H1 and Man H6 (FIG. 21). Therefore, the NMR results support the linkage analysis data, both confirmed the major component of Prolectin-M is galactomannan.

TABLE 4
Chemical shift assignment of major glycosyl residues of Prolectin-M.

| Glycosyl | C/H-1 | C/H-2 | C/H-3 | C/H-4 | C/H-5 | C/H-6 |
|---|---|---|---|---|---|---|
| α-Man_{Red} | 94.06/5.2 | 71.06/4.0 | 72.99/3.9 | 77.11/3.8 | — | — |
| β-Man_{Red} | 93.76/4.9 | 68.50/4.0 | — | — | — | — |
| t-α-Gal | 98.88/5.0 | 68.48/3.9 | 69.45/4.0 | 69.35/4.0 | 71.37/3.9 | 61.31/3.8 |
| 4-β-Man | 100.20/4.8 | 69.99/4.2 | 71.53/3.8 | 76.96/3.9 | 75.11/3.6 | 60.60/3.9, 3.8 |
| 4,6-β-Man | 100.20/4.8 | 69.99/4.2 | 70.99/3.8 | 77.10/3.9 | 73.34/3.8 | 66.53/4.0, 3.8 |
| t-β-Man | 100.20/4.8 | 70.57/4.1 | 73.08/3.7 | 66.71/3.6 | 76.26/3.4 | 62.92/3.9, 3.8 |

Prolectin-M used in NMR analysis was the supernatant or easily-soluble portion of Prolectin-M, i.e., galactomannan molecules with lower molecular sizes. This agreed with the results from the linkage analysis and with the observation in the 1D ¹H-NMR spectrum of relatively large peaks for the reducing end Man residues.

### Example 4: NMR Analysis of Prolectin-I

### Glycosyl Linkage Analysis

Prolectin-I solution from two sample vials (total 40 mL) was dialyzed in a dialysis tubing (COMW, 3.5 kDa) against nanopure water (total 4×3 L) for 2 days. The dialyzed sample was lyophilized. Glycosyl linkage analysis was performed by combined gas chromatography/mass spectrometry (GC/MS) of the partially methylated alditol acetates (PMAAs) derivatives produced from the sample. The procedure was a slight modification of the one described by Willis et al. (2013) PNAS, 110 (19) 7868-7873.

Briefly, methylation of the sample using dimsyl potassium base was performed. This was followed by acetylation using N-methylimidazole and acetic anhydride. The sample was extracted with dichloromethane, and the carboxylic acid methyl esters were reduced using lithium aluminum deuteride in THF (80° C., 8 h). After desalting using an On-guard H⁺ column (ThermoFisher), the sample was remethylated using two rounds of treatment with sodium hydroxide (15 min each) and methyl iodide (45 min each). The sample was then hydrolyzed using 2 M TFA (2 h in sealed tube at 120° C.), reduced with NaBD₄, and acetylated using acetic anhydride/TFA. The resulting PMAAs were analyzed on an Agilent 7890A GC interfaced to a 5975C MSD (mass selective detector, electron impact ionization mode); separation was performed on a 30 m Supelco SP-2331 bonded phase fused silica capillary column.

TABLE 5
GC temperature program for linkage analysis

| | Rate (° C./min) | Value (° C.) | Hold Time (min) |
|---|---|---|---|
| Initial | | 60 | 1 |
| Ramp1 | 27.5 | 170 | 0 |
| Ramp2 | 4 | 235 | 2 |
| Ramp3 | 3 | 240 | 12 |

### NMR Analysis

Sample Prolectin-I 7.24 mg was weighed and dissolved in 600 μl of D₂O. The supernatant was transferred into an NMR tube. Ten microliters of 1 mM DSS was added into the NMR tube as internal standard. The sample was analyzed at 25° C. with a Bruker 900 MHZ NMR instrument equipped with a cryoprobe. A standard zgf2pr pulse sequence was employed with a pre-saturation sequence for water suppression. Pulse sequences, hsqcetgpsisp2 and clmlevphpr, were applied for collecting HSQC and TOCSY spectrum, respectively.

### Results

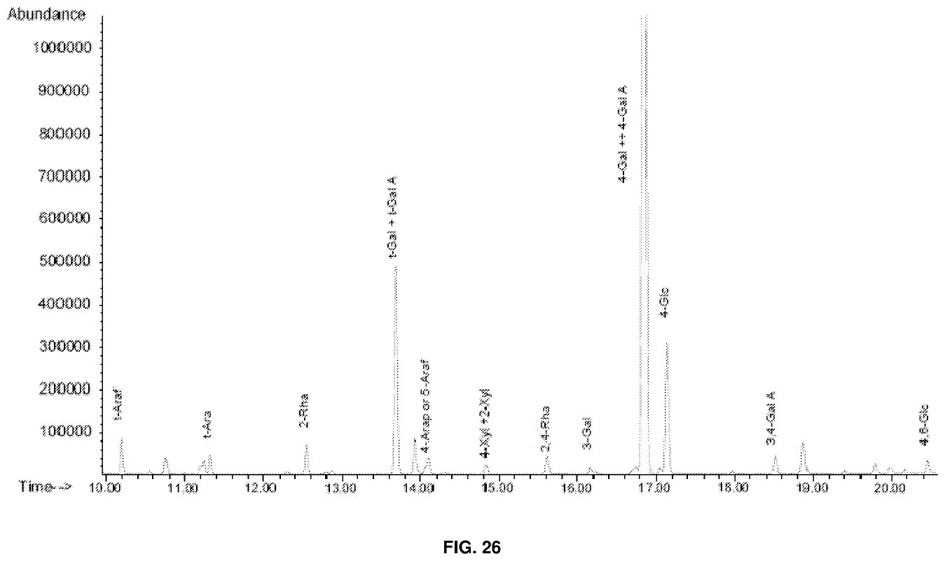

The glycosyl linkage analysis chromatogram is shown in FIG. 26, and the results are listed in Table 6. In summary, the most abundant glycosyl residues of Prolectin-I were GalpA residues (45.7%), including 4-GalpA (34.9%), 3,4-GalpA (1.3%), and t-GalpA (9.5%). In addition, 2-Rhap & 2,4-Rhap residues accounted for 3.4% of the glycosyl residues. This suggested that Prolectin-I has a pectin structure, which is mainly composed of homogalacturonan (HG) with short rhamnogalacturonan-I (RG-I) fragment(s). Moreover, the presence of t-Galp (5.7%) and 4-Galp (30.0%) indicated that the RG-I part of Prolectin-I pectin has β-1,4-galactan side chains. Other glycosyl residues such as t-Araf, t-Arap, 4-Arap/5-Araf, and 3,6-Galp could be minor substituted glycosyl residues.

TABLE 6
Relative percentage of each detected linkage in Prolectin-I.

| | Relative Area |
|---|---|
| Glycosyl Linkage Residue | Percentage (%) |
| Terminal Arabinofuranosyl Residue (t-Araf) | 1.9 |
| Terminal Arabinopyranosyl Residue (t-Arap) | 1.1 |
| 2-linked Rhamnopyranosyl Residue (2-Rhap) | 2.0 |
| Terminal Galactopyranosyl Residue (t-Galp) | 5.7 |
| Terminal Galactopyranosyl Uronic Acid Residue (t- | 9.5 |
| GalpA) | |
| 4-linked Arabinopyranosyl or 5-linked | 1.0 |
| Arabinofuranosyl Residue (4-Arap or 5-Araf) | |
| 2-linked Xylopyranosyl Residue (2-Xylp) | 0.4 |
| 4-linked Xylopyranosyl Residue (4-Xylp) | 0.2 |
| 2,4-linked Rhamnopyranosyl Residue (2,4-Rhap) | 1.4 |
| 3-linked Galactopyranosyl Residue (3-Galp) | 0.4 |
| 4-linked Galactopyranosyl Residue (4-Galp) | 30.0 |
| 4-linked Galactopyranosyl Uronic Acid Residue (4- | 34.9 |
| GalpA) | |
| 4-linked Glucopyranosyl Residue (4-Glcp) | 9.6 |
| 3,4-linked Galactopyranosyl Uronic Acid Residue | 1.3 |
| (3,4-GalpA) | |
| 3,6-linked Galactopyranosyl Residue (3,6-Galp) | 0.7 |
| Total | 100.1 |

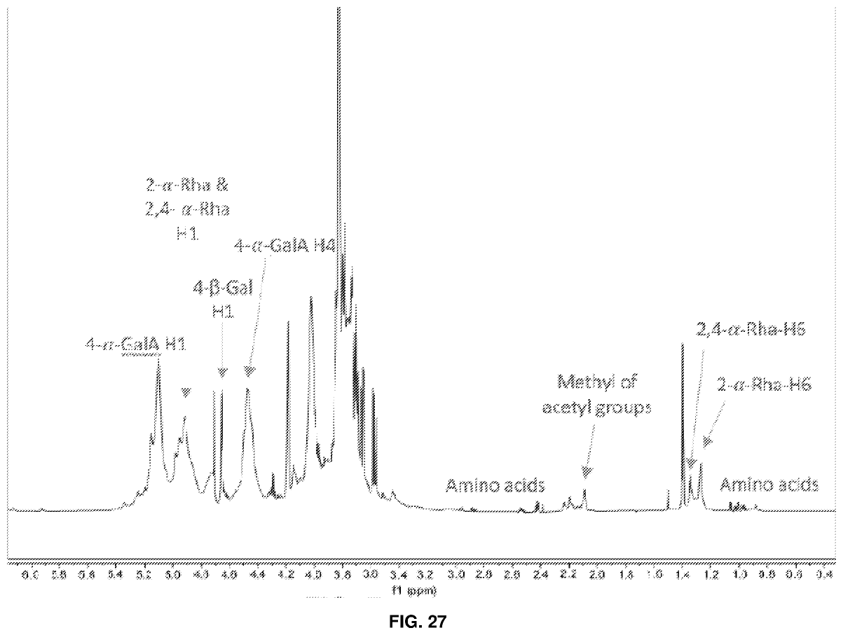

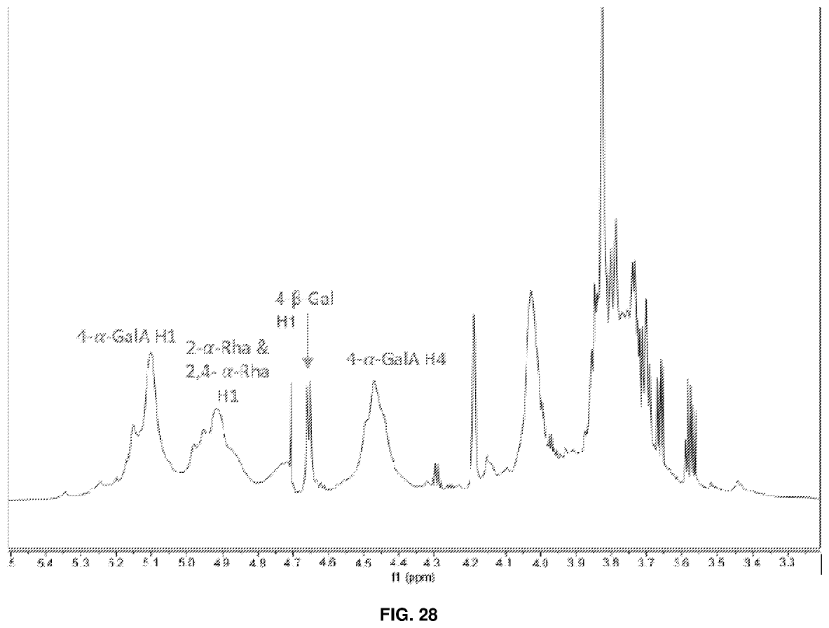

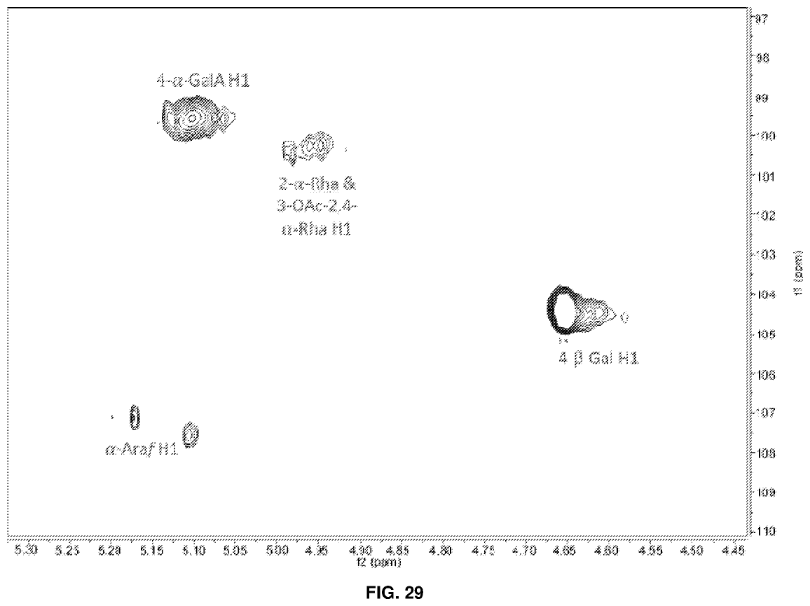

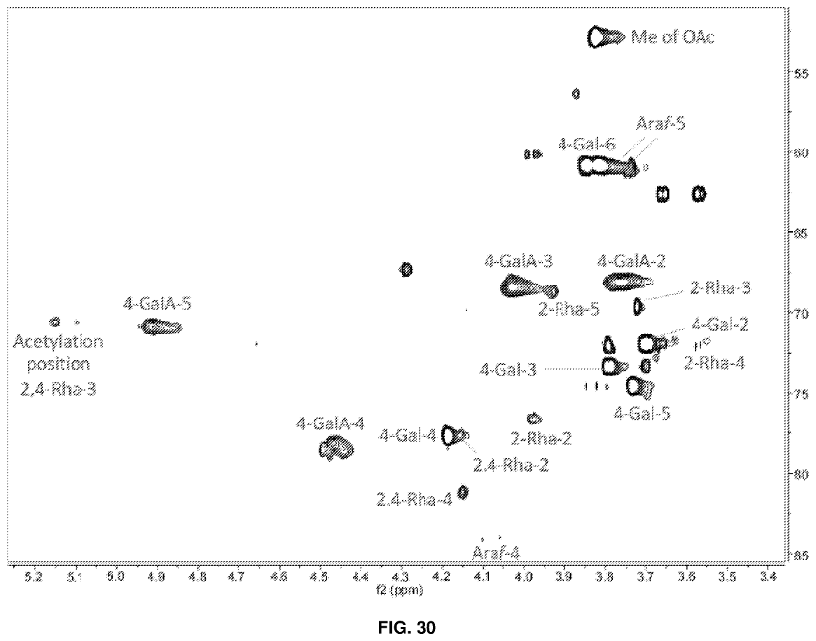

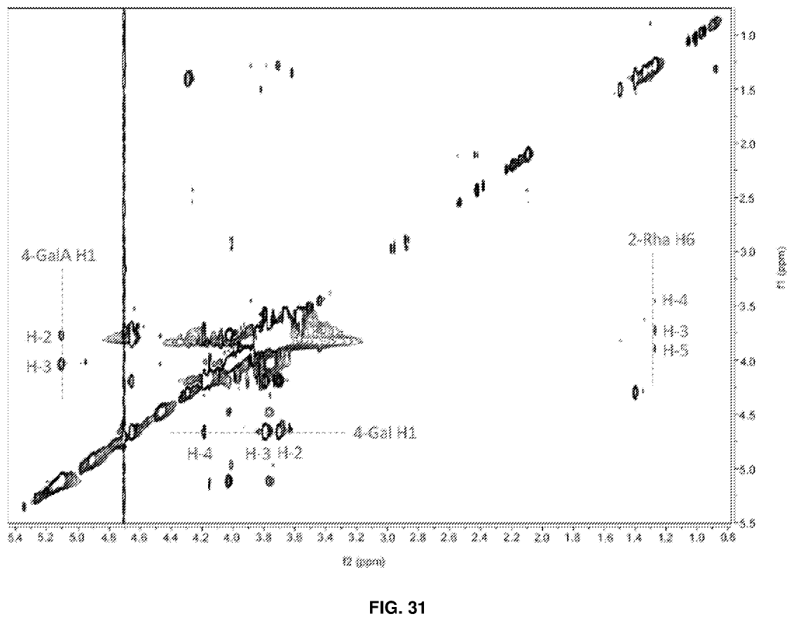

The NMR spectra are shown in FIGS. 27-31. The 1D ¹H-NMR spectrum showed that the GalA and Rha had alpha-configurations, while Gal residues have beta-configurations (FIGS. 27 and 28). The ¹H/¹³C cross-peaks were assigned and labeled in FIG. 29 and FIG. 30. The chemical shifts of the major glycosyl residues are summarized in Table 7. The chemical shift assignments were based on a TOCSY spectrum (FIG. 31) and published data (see references below). In summary, the NMR results supported the linkage analysis data; both confirmed the major component of Prolectin-I is pectin, composed of HG and β-1,4-galactan-containing RG-I.

TABLE 7
Chemical shift assignment of major glycosyl residues of Prolectin-I.

| Glycosyl | C/H-1 | C/H-2 | C/H-3 | C/H-4 | C/H-5 | C/H-6 |
|---|---|---|---|---|---|---|
| α-Araf | 107/5.17 | n.d. | n.d. | 84.1/4.10 | n.d. | — |
| α-Araf | 107/5.11 | n.d. | n.d. | 84.1/4.06 | n.d. | — |
| 4-α-GalA | 99.6/5.10 | 68.0/3.76 | 68.5/4.03 | 78.2/4.47 | 70.9/4.91 | — |
| 2-α-Rha | 100/4.96 | 76.6/3.98 | 69.7/3.72 | 71.8/3.55 | 68.7/3.94 | 16.6/1.26 |
| 3-OAc- | 100/4.96 | 77.7/4.15 | 70.6/5.15 | 81.1/4.15 | — | 19.7/1.34 |
| 2,4-α-Rha | | | (OAc: 52.9/3.82) | | | |
| 4-β-Gal | 104/4.66 | 71.9/3.70 | 73.4/3.79 | 77.7/4.19 | 74.6/3.73 | 60.9/3.85, 3.81 |

Prolectin-I also contained weak signals of amino acids (FIG. 27 and FIG. 30), as well as about 9.6% of 4-Glcp as identified in the glycosyl linkage analysis (Table 6).

Other embodiments are in the claims.

1. A method of treating a viral infection in a subject in need thereof, the method comprising administering to the subject an effective amount of lectin-binding carbohydrates.

2. The method of claim 1, wherein the lectin-binding carbohydrates are pectin polysaccharides.

3. (canceled)

4. The method of claim 1, wherein the lectin-binding carbohydrates are galactomannans.

5. (canceled)

6. The mixture of claim 4, wherein the lectin-binding carbohydrates are a mixture of fenugreek galactomannans and guar galactomannans.

7. The method of claim 1, wherein the lectin-binding carbohydrates are polysaccharides that comprise N-acetylglucosamine and mannose or N-acetylglucosamine and mannose and at least one of galactose or N-acetylneuraminic acid.

8. (canceled)

9. The method of claim 1, wherein the lectin-binding carbohydrates are glycosaminoglycans.

10. (canceled)

11. The method of claim 1, wherein the lectin-binding carbohydrates are glycolipids.

12. The method of claim 11, wherein the lectin-binding carbohydrates are glycosphingolipids.

13. (canceled)

14. The method of claim 1, wherein the lectin-binding carbohydrates are polylactosamines.

15. The method of claim 1, wherein the lectin-binding carbohydrate comprises sialic acid.

16. The method of claim 1, wherein the viral infection is caused by a retrovirus, a human norovirus infection, a herpesvirus, a coronavirus, an orthomyxovirus, an adenovirus, a flavivirus, a rotavirus, a mononegavirus, or a rubella virus infection.

17. The method of claim 16, wherein the viral infection is caused by human immunodeficiency virus, human T-lymphotropic virus type 1, or human T-lymphotropic virus type 2.

18-19. (canceled)

20. The method of claim 16, wherein the viral infection is caused by herpes simplex virus 1, herpes simplex virus 2, varicella zoster virus, human cytomegalovirus, Epstein-Barr virus, roseolovirus, pseudorabies virus, or Kaposi's sarcoma associated herpesvirus.

21. (canceled)

22. The method of claim 16, wherein the viral infection is caused by SARS-CoV-2, SARS-CoV-1, MERS-CoV, human coronavirus 229E, human coronavirus NL63, human coronavirus OC43, or human coronavirus HKU1.

23. The method of claim 22, wherein the viral infection is caused by SARS-CoV-2.

24. (canceled)

25. The method of claim 16, wherein the viral infection is an orthomyxovirus is selected from: an alphainfluenzavirus, betainfluenzavirus, deltainfluenzavirus, gammainfluenzavirus, isavirus, thogotovirus, or quaranjavirus.

26. (canceled)

27. The method of claim 16, wherein the viral infection is caused by human adenovirus A, human adenovirus B, human adenovirus C, human adenovirus D, human adenovirus E, human adenovirus F, or human adenovirus G, West Nile virus, dengue virus, tick-born encephalitis virus, yellow fever virus, Zika virus, hepatitis C virus, Murray Valley encephalitis virus, Tick-borne encephalitis virus, Saint Louis encephalitis virus, Japanese encephalitis virus, rotavirus A, rotavirus B, rotavirus C, rotavirus D, rotavirus F, rotavirus G, rotavirus H, rotavirus I, rotavirus J, Ebola virus, Marburg virus, measles virus, mumps virus, Nipah virus, or rabies virus.

28-32. (canceled)

33. The method of claim 16, wherein the viral infection is caused by an orthopneumovirus selected from: human metapneumovirus, human respiratory syncytial virus A2, or human respiratory syncytial virus B1.

34-36. (canceled)

37. The method of claim 1, wherein the lectin-binding carbohydrates are formulated in a pharmaceutical composition with a pharmaceutically acceptable carrier.

38. (canceled)

39. The method of claim 37, wherein the pharmaceutical composition is a chewable tablet.

40. (canceled)
